SEQUENCE LISTING (160 sequences)

SEQ ID NO: 1 - protein, 510 aa, Mus musculus
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 2 - DNA, 12 bp, Artificial Sequence
  Test Polynucleotide p875
cagttaaaaact

SEQ ID NO: 3 - DNA, 11 bp, Artificial Sequence
  Test Polynucleotide p876
gagttaaaact

SEQ ID NO: 4 - DNA, 10 bp, Artificial Sequence
  Test Polynucleotide p877
cagcaaggct

SEQ ID NO: 5 - DNA, 18 bp, Artificial Sequence
  primer
aaaaaaaaaaaaaagggg

SEQ ID NO: 6 - protein, 25 aa, Artificial Sequence
  affinity tag for purifying TdT variants
MASSHHHHHHSSGSENLYFQTGSSG

SEQ ID NO: 7 - protein, 381 aa, Mus musculus
NSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 8 - protein, 380 aa, Unknown
  Bovine
DYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 9 - protein, 380 aa, Homo sapiens
DYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 10 - protein, 376 aa, Gallus domesticus
QYPTLKTPESEVSSFTASKVSQYSCQRKTTLNNCNKKFTDAFEIMAENYEFKENEIFCLEFLRAASVLKSLPFPVTRMKDIQGLPCMGDRVRDVIEEIIEEGESSRAKDVLNDERYKSFKEFTSVFGVGVKTSEKWFRMGLRTVEEVKADKTLKLSKMQRAGFLYYEDLVSCVSKAEADAVSSIVKNTVCTFLPDALVTITGGFRRGKKIGHDIDFLITSPGQREDDELLHKGLLLYCDIIESTFVKEQIPSRHVDAMDHFQKCFAILKLYQPRVDNSSYNMSKKCDMAEVKDWKAIRVDLVITPFEQYAYALLGWTGSRQFGRDLRRYATHERKMMLDNHALYDKRKRVFLKAGSEEEIFAHLGLDYVEPWERNA

SEQ ID NO: 11 - protein, 387 aa, Didelphis marsupialis
SANPDPTAGTLNILPPTTKTISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYATHEKKMMLDNHALYDKTKKIFLKAKSEEEIFAHLGLEYIQPSERNA

SEQ ID NO: 12 - protein, 381 aa, Unknown
  shrew
DCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 13 - protein, 387 aa, Unknown
  python
EKYQLPEDEDRSVTSDLDRDSISEYACQRRTTLKNYNQKFTDAFEILAENYEFNENKGFCTAFRRAASVLKCLPFTIVQVHDIEGVPWMGKQVKGIIEDIIEEGESSKVKAVLDNENYRSVKLFTSVFGVGLKTSDKWYRMGLRTLEEVKRDKNLKLTRMQKAGFLHYDDLTSCVSKAEADAASLIVQDVVWKIVPNAIVTIAGGFRRGKQTGHDVDFLITVPGSKQEEEELLHTVIDIWKKQELLLYYDLIESTFEDTKLPSRKVDALDHFQKCFAILKVHKEREDKGNSIRSKAFSEEEIKDWKAIRVDLVVVPFEQYAFALLGWTGSTQFERDLRRYATHEKKMMLDNHALYDKTKKIFLNAASEEEIFAHLGLDYLEPWERNA

SEQ ID NO: 14 - protein, 381 aa, Canis familiaris
DYTASPNPELQKTLPVAVKKISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 15 - protein, 382 aa, Unknown
  mole
GDCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 16 - protein, 379 aa, Ochotona princeps
EYSANPSPGPQATPAVYKISQYACQRRTTLNNHNHIFTDAFEILAENYEFKENEGCYVTYMRAASVLKSLPFTIVSMKDTEGIPCLEDKVKSIMEEIIEEGESSEVKAVLSDERYQCFKLFTSVFGVGLKTSEKWFRMGFRSLSNIRLDKSLKFTQMQKAGFRYYEDIVSCVTRAEAEAVDVLVNEAVRAFLPDAFITMTGGFRRGKKIGHDVDFLITSPELTEEDEQQLLHKVMNLWEKKGLLLYHDLVESTFEKLKQPSRKVDALDHFQKCFLIFKLYHERVGGDRCRQPEGKDWKAIRVDLVMCPYECHAFALLGWTGSRQFERDLRRYASHERKMILDNHALYDKTKRVFLQAENEEEIFAHLGLDYIEPWERNA

SEQ ID NO: 17 - protein, 384 aa, Erinaceus europaeus
DASFGSNPGSQNTPPLAIKKISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRQFERDLRRFATHERKMMLDNHALYDKTKRIFLKAESEEEIFAHLGLDYIDPWERNA

SEQ ID NO: 18 - protein, 381 aa, Unknown
  tree shrew
DHSTSPSPGPQKTPALAVQKISQYACQRRTTLNNCNRVFTDAFETLAENYEFRENEDSSVIFLRAASVLRSLPFTITSMRDTEGLPCLGDKVKCVIEEIIEDGESSEVNAVLNDERYKSFKLFTSVFGVGLKTSEKWFRMGFRTLSRVRSDKSLHLTRMQQAGFLYYEDLASCVTRAEAEAVGVLVKEAVGAFLPDALVTITGGFRRGKKTGHDVDFLITSPGSTEEKEEELLQKVLNLWEKKGLLLYYDLVESTFEKLKTPSRKVDALDHFPKCFLILKLHHQRVDGDKPSQQEGKSWKAIRVDLVMCPYERHAFALLGWTGSRQFERDLRRYATHERKMMLDNHALYDKTKRVFLKAESEEDIFAHLGLDYIEPWERNA

SEQ ID NO: 19 - protein, 394 aa, Ornithorhynchus anatinus
LTNSAPINCMTETPSLATKQVSQYACERRTTLNNCNQKFTDAFEILAKDFEFRENEGICLAFMRAISVLKCLPFTIVRMKDIEGVPWLGDQVKSIIEEIIEDGESSSVKAVLNDERYRSFQLFNSVFEVGLTDNGENGIARGFQTLNEVITDENISLTKTTLSTSLWNYLPGFLYYEDLVSCVAKEEADAVYLIVKEAVRAFLPEALVTLTGGFRRGKKIGHDVDFLISDPESGQDEQLLPNIIKLWEKQELLLYYDLVESTFEKTKIPSRKVDAMDHFQKCFLILKLHHQKVDSGRYKPPPESKNHEAKNWKAIRVDLVMCPFEQYAYALLGWTGSRQFERDLRRYATHEKKMMLDNHALYDKTKKIFLKAESEEDIFTHLGLDYIEPWERNA

SEQ ID NO: 20 - protein, 384 aa, Jaculus blanfordi
SSELELLDVSWLIECMGAGKPVEMTGRHQLVKQTFCLPGFILQDAFDILAENCEFRENEASCVEFMRAASVLKSLPFPIISVKDTEGIPWLGGKVKCVIEEIIEDGESSEVKALLNDERYKSFKLFTSVFGVGLKTAERWFRMGFRTLSTVKLDKSLTFTRMQKAGFLHYEDLVSCVTRAEAEAVSVLVQQAVVAFLPDALVSMTGGFRRGKKIGHDVDFLITSPEATEEEEQQLLHKVTNFWEQKGLLLYCDHVESTFEKCKLPSRKVDALDHFQKCFLILKLYRERVDSVKSSQQEGKGWKAIRVDLVMCPYECRAFALLGWTGSRQFERDLRRYATHERKMRLDNHALYDKTKRVFLKAESEEEIFAHLGLEYIEPLERNA

SEQ ID NO: 21 - protein, 364 aa, Unknown
  Puma
VVKKIPLYACQRRTTLNNFNHIFTDAFEVLAENYEFKENEISSATFMRAASVLKLPFTIISMKDTEGIPCLGDKVKCVIEEIIEDGESSEVKAVLNDERYQSFKLFTSVFGVGLKTSEKWFRMGFRTLSKIKSDKTLKFTQMQKAGFLYYEDLVSCVTRAEAEAVGVLVKEAVWAFLPDAFVTMTGGFRRGKKIGHDVDFLITIPGSTDEEEEQLLPKVINLWQRKELLLYYDLVESTFEKLKLPSRKVDALDHFQKCFLILKLHHQRVDSGKCSQQEGKTWKAIRVDLVMCPYERRAFALLGWTGSRQFERDLRRYATHERKMILDNHALYDKTKKIFLKAESEEEIFAHLGLDYIEPWERNA

SEQ ID NO: 22 - protein, 362 aa, Artificial Sequence
  N139 reptilian
YACQRRTTLNNYNKKYTDAFEILAENYEMRENAGACLAFRRAASVLKFLPFAIVRSNDIEGLPWMGEQVKGIIEDILEEGQSPKVEAVLNNEKYRSFKLFTSVFGVALKTSEKWFMMGLRNLEDVKLNQNLQLTRMQKAGLQHYEDLISYVSKAEADSTSLMVKDTVWKFSPSALVTLTGGFRRGKKMGHDVDFLITVPGSRPNEELLHLVIDCWKKQGLLLYYDLIESTFEKSKLPSQRVDALDHFQKCFAILKLHKERVNQGTSLPPVASTVEEIKDWKAIRVDLVVSPFEQHAFALLGWTGSRQFERDLRRYATHEKKMMLDNHALYDKTKKIFLSASSEEEIFAHLGLDYLEPWERNA

SEQ ID NO: 23 - protein, 364 aa, Unknown
  shrew
VQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 24 - protein, 382 aa, Serinus canaria
SPPLNTPELEMPSFIATKVSQYSCQRKTTLNNYNKKFTDAFEVMAENYEFKENEIFCLEFLRAASLLKSLPFSVTRMKDIQGLPCMGDQVRDVIEIIEEGESSRVKEVLNDERYKAFKQFTSVFGVGVKTSEKWYRMGLRTVGEVKADKTLKLSKMQKAGFLYYEDLVSCVSKAEADAVSLIVKNTVCTFLPDALVTITGGFRRGKNIGHDIDFLITNPGPREDDELLHKVIDLWKKQGLLLYCDIIESTFVKEQLPSRKIDAMDHFQKCFAILKLYQPRVDNSTCNTSKKLEMAEVKDWKAIRVDLVITPFEQYSYALLGWTGSRQFGRDLRRYAAHERRMILDNHGLYDRTKRIFLKAGSEEEIFAHLGLDYVEPWERNA

SEQ ID NO: 25 - protein, 370 aa, Neopelma chrysocephalum
FIARKVSQYSCQRKTTLNNYNKKFTDAFEIMAENYEFKENEIFCLEFLRAASLLKYLPFPVTRMKDIQGLPCIGDQVRDVIEGIIEEGESSRVKEVLNDERYKAFKQFTSVFGVGVKTSEKWYRMGLRTVEELKADKTLKLSKMQKAGFLYYEDLVSCVSKAEADAVTLIVKNTVSTFLPDALVTITGGFRRGKKMGHDIDFLITNPGPREDDELLHKVVDLWKKQGLLLYCDIIESTFVEEQLPSRKVDAMDNFQKCFTILKLYQPGVDNSSYNMSKKSDMAEVKDWKAIRVDLVITPFEQYAYALLGWTGSREFGRDLRRYASHERKMILDNHGLYDRRKRIFLKAGSEEEIFAHLGLDYVEPWERNA

SEQ ID NO: 26 - protein, 383 aa, Alligator sinensis
SPSPVPGSQNVPAPSVDKVSQYACQRRTTLNNYNKKFTDAFEILAENCEFRENRLGCLEFLRAASVLKFLPFPIVKMKNIEGLPCMGDKVKCVIEEILEEGESCQAKEILNDERYKSFKLFTSVFGVGLKTTEKWYRMGFRTLEEVKAEKTLKLSRMQIAGFLHYEDIISYVSKAEADAVSLLIKDTVCMFLPDALVTITGGFRRGKKTGHDVDFLITNPGPEEEKELLHKVVDLWEKQGLLLYYDVIESTFEKEKRPSRKVDALDHFQKCFAILKLHQQRRGNSNSNISKESDKAEVKDWKAIRVDLVISPFEQYAYALLGWTGSRQFERDLRRYASRERKMMLDNHALYDKTKRTFLKAESEEEIFAHLGLDYIEPWERNA

SEQ ID NO: 27 - protein, 380 aa, Xenopus laevis
SPSPVPGSQNVPAPSEVKVSQYACQRCTTLQDTNRIFTDAFDILAEHFEFCENKGRTVAFLRASSLIKSLPFPITAMKELEGLPWLGDQMKGIIEEILEEGKSYKVLEVMNEERYKSFKQFTSVFGVGLKTSDKWFRMGFRTLEEIKNEKELKLTKMQKCGLLYYEDITSYVSRAEAETTEQLIKSIVWKFVPDAIVTLTGGFRRGKKKGHDVDILITCARKGKEKNILHNTMSVLKNRGLLLFYNIIESTFDETKLPSRHVDALDHFQKCFTILKLPKRQMDIGNIIDPHECERKNWKAVRLDLVITPYEQYPYALLGWTGSRQFERDLRRYATHEKRMMLDNHGLYDKTKNNFLKANNEEDIFKQLGLDYLEPWERNA

SEQ ID NO: 28 - protein, 383 aa, Notechis scutatus
SPSPVPGSQNVPAPSVDSISPYACQRRTTLKNYNQKFTDAFEILVENYEFNENKEFCLAFGRAASLLKCLPFTVVRVNDIEGLPWMGKQVREIIEDILEEGESSKVKAVLNDENYRSIKLFTSVFGVGLRTSEKWYRMGLRTLEEVKCNKNLTLTRMQKAGFFYYDDLISSVSKAEADAATQIVQDTVWKILPNAVVTLTGGFRRGKQTGHDVDFLITVPGSRQEEELLHPVIDIWKKQELLLYYDLIESTFENTKLPSRKVDALDHFQKCFAILKVHKERVNKGSAVQSNVFAEEGTKDWKAIRVDLVVTPFQHYAFALLGWTGSRQFERDLRRYATQEKKMMLDNHALYDKTKKIFLSAANEEEIFSHLGLDYLEPWERNA

SEQ ID NO: 29 - protein, 367 aa, Salmo truttal
TAAANVSQYACLRRTTTENHNKIFTDVLEELAENSEFNESKGPCLAFRRAASVLKSLPSAVHCLGAIQGLPCLGEHTKAVMEEILIFGRSFKVEEVQSDERYQALKLFTSVFGVGPKTAEKWYRRGLRSLKEILAEPNIQLNRMQRAGFLYYRDISKAVSKAEAKALSSIIEETAHWIAPDSILALTGGFRRGKEYGHDVDFLLTMPEMGKEEGLLLRVIDRLRDQGILLYCEHQDSTFDMSKLPSHRFEAMDHFEKCFLILRLEEGQVEGDGGLQKDPGESRGWRAVRVDLVAPPVDRYAFVLLGWTGSRQFERDLRRFASKERGMCLDNHALYDKTKKLFLPATSEEDIFAHLGLEYVEPWQRNA

SEQ ID NO: 30 - protein, 377 aa, Electrophorus electricus
SPSPVPGSQNVPAPSLPTVSQYACQRRTTLDNHNKVFTDALEVLIENYEFSDNKGACVGFRRAASVLKSLPKPLRCLKDMEGLPCLGDDTKAIIEEIYECGSSSRVENILSDEKYQTLKLFTSVFGVGPKTGEKWYRRGLRALEQVHSEPSIQLNKMQAAGFLYYEDISKPVSRAEAKAVGCIIEEVASCFSSSVTITLTGGFRRGKEFGHDVDFLLSIPEPGKEDGLLPAVIDRLRKQGILLYSDLQESTLQQWKRPSRCFDSMDHFQKCFLIVKLWTRLVEGHREDPSSQRDWKAVRVDLVVPPVDCYAFALLGWSGSTQFERDLRRFARLERRMLLDNHALYDKTTNTFLQAKTEEDIFAHLGLDYIEPWQRNA

SEQ ID NO: 31 - protein, 378 aa, Anabas testudineus
SPSPVPGSQNVPAPSVATVSQYACQRRTTTENNNKILTDAFEVLAESYELNQLEGPCLAFRRAASVLKSLPWAVQCLGATQGLPCLGEHTKALIEEILQYGHSFEVEKILSDERYQTLKLFTSVFGVGPKTAEKWYRRGLRSFSDILAEPSIQLNRMQQSGFLHYGDISRAVSKAEARALGNIIDEAVHAITPDGILALTGGFRRGKEFGHDVDFIVTTPEQGKEETLLPNIIDRLKEQGILLYSDYQTSTFDISKLPSHKFEAMDHFAKCFLILRLEGSLVDRGLNSTEGDSRGWRAVRVDLVSPPMERYAYALLGWTGSRQFERDLRRFARLEQHMLLDNHALYDKTKKEFLAATTERDIFAHLGLEYIEPWQRNA

SEQ ID NO: 32 - protein, 378 aa, Poecilia reticulate
SPSPVPGSQNVPAPSVDKVSQYACQRRTTVENNNRIFTDAFEVLAENYEFNEIEGRCLAFRRAASVLKSLPWAVRSVGATLDLPCLGEHTTAVMKEILQYGRSFEVEKILSDERCQTLKLFTSVFGVGPKTAEKWYRRGLRSFSDVLAQPGIHLNRMQQSGFLHYGDISRAVSKAEARAVGNIIDEAVHVITPNAILALTGGFRRGKDFGHDVDFIVTTTELGKEKNLLISVIESLKKQGLLLFSDYQASTFDISKLPSHRFEAMDHFAKCFLILRLEGSRVEGGLQRAQADGRGWRAVRVDLVSPPADRFAFTMLGWTGSRMFERDLRRFARLERQMLLDNHALYDKTKKEFLTAATEKDIFDHLGLEYIEPWQRNA

SEQ ID NO: 33 - protein, 366 aa, Rattus norvegicus
PLMQKISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 34 - protein, 379 aa, Rattus norvegicus
SLSPVPGSQTVPPPLMQKISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 35 - protein, 379 aa, Piliocolobus tephrosceles
SDSTNPGLPKTPPTAIQKISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSALVKEAVWAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSDQSSWQGGKTWKAIRVDLVMCPYERRAFALLGWTGSRQFERDLRRYATHERKMILDNHALYDKTKRIFLKAESEEEIFAHLGLDYIEPWERNA

SEQ ID NO: 36 - protein, 349 aa, Sus scrofa
SASPSPGSQNTLPPAVKKISQYACQRRTTLNNCNHIFTDAFEVLAENYEFRENETFCLAFMRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDK

SEQ ID NO: 37 - protein, 379 aa, Panthera tigris altaica
YSASPNPELQKTPPLVKKIPLYACQRRTTLNNFNHVFTDAFEVLAENYEFKENEVSSATFMRAASVLKSLPFTIISMKDTEGIPCLGDKVKCVIEEIIEDGESSEVKAVLNDERYQSFKLFTSVFGVGLKTSEKWFRMGFRTLSKIKSDKTLKFTQMQKAGFLYYEDLVSCVTRAEAEAVGVLVKEAVWAFLPDAFVTMTGGFRRGKKIGHDVDFLITSPGSTDEEEEELLPKVINLWQRKELLLYYDLVESTFEKLKLPSRKVDALDHFQKCFLILKLHHQRVDSGKCSQQEGKTWKAIRVDLVMCPYERRAFALLGWTGSRQFERDLRRYATHERKMILDNHALYDKTKKIFLKAESEEEIFAHLGLDYIEPWERNA

SEQ ID NO: 38 - protein, 365 aa, Bubalus bubalis
LAVKKISQYACQRKTTLNNYNHIFTDAFEILAENSEFKENEVSYVTFMRAASVLKSLPFTIISMKDTQGIPCLGDKVKCVIEEIIEDGESSEVKAVLNDERYQSFKLFTSVFGVGLKTSEKWFRMGFRSLSKITSDKTLKFTKMQKAGFLYYEDLVSCVTRAEAEAVGVLVKEAVWAFLPDAFITMTGGFRRGKKIGHDVDFLITSPGSAEDEEQLLPKVINLWEKKGLLLYYDLVESTFEKFKLPSRQVDTLDHFQKCFLILKLHHQRVDSGRSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 39 - protein, 379 aa, Marmota flaviventris
PDHSNSDPQRIPPPAVQTISQYACQRRTTLNNCNRVFTDAFDVLAENYEFRENESCSVVFMRAASVLKSLPFTIISMRDLEGIPCLEGKAKSIIEEIIEDGESSEVKAVLNDERYKSFKLFTSVFGVGLKTSEKWFRMGFRTLSKIRSDKSLKFTHMQKAGFLYYEDLVSCVTRAEAEAVSVLVKEAVWAFLPDAFITMTGGFRRGKNIGHDVDFLITSAEATEEEEQQLLHKVTNLWEKKGLLLYCDLVESTFEKLKTPSRKVDALDHFQKCFLILKLHHQRVDSDKASQQGGKNWKAIRVDLVMCPYERRAFALLGWTGSRQFERDLRRYATHERKMILDNHALYDKTKRIFLKAESEEEIFAHLGLDYIEPWERNA

SEQ ID NO: 40 - protein, 362 aa, Mus musculus
KISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 41 - protein, 361 aa, Unknown
  Bovine
KISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 42 - protein, 361 aa, Homo sapiens
KISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 43 - protein, 358 aa, Gallus domesticus
KVSQYSCQRKTTLNNCNKKFTDAFEIMAENYEFKENEIFCLEFLRAASVLKSLPFPVTRMKDIQGLPCMGDRVRDVIEEIIEEGESSRAKDVLNDERYKSFKEFTSVFGVGVKTSEKWFRMGLRTVEEVKADKTLKLSKMQRAGFLYYEDLVSCVSKAEADAVSSIVKNTVCTFLPDALVTITGGFRRGKKIGHDIDFLITSPGQREDDELLHKGLLLYCDIIESTFVKEQIPSRHVDAMDHFQKCFAILKLYQPRVDNSSYNMSKKCDMAEVKDWKAIRVDLVITPFEQYAYALLGWTGSRQFGRDLRRYATHERKMMLDNHALYDKRKRVFLKAGSEEEIFAHLGLDYVEPWERNA

SEQ ID NO: 44 - protein, 368 aa, Artificial Sequence
  Possum truncated  M63 C173 R207 R331 E334
TISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYATHEKKMMLDNHALYDKTKKIFLKAKSEEEIFAHLGLEYIQPSERNA

SEQ ID NO: 45 - protein, 362 aa, Unknown
  shrew
KISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 46 - protein, 362 aa, Canis familiaris
KISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 47 - protein, 362 aa, Unknown; identical to SEQ ID NO: 45
  mole

SEQ ID NO: 48 - protein, 362 aa, Ochotona princeps
KISQYACQRRTTLNNHNHIFTDAFEILAENYEFKENEGCYVTYMRAASVLKSLPFTIVSMKDTEGIPCLEDKVKSIMEEIIEEGESSEVKAVLSDERYQCFKLFTSVFGVGLKTSEKWFRMGFRSLSNIRLDKSLKFTQMQKAGFRYYEDIVSCVTRAEAEAVDVLVNEAVRAFLPDAFITMTGGFRRGKKIGHDVDFLITSPELTEEDEQQLLHKVMNLWEKKGLLLYHDLVESTFEKLKQPSRKVDALDHFQKCFLIFKLYHERVGGDRCRQPEGKDWKAIRVDLVMCPYECHAFALLGWTGSRQFERDLRRYASHERKMILDNHALYDKTKRVFLQAENEEEIFAHLGLDYIEPWERNA

SEQ ID NO: 49 - protein, 365 aa, Erinaceus europaeus
KISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRQFERDLRRFATHERKMMLDNHALYDKTKRIFLKAESEEEIFAHLGLDYIDPWERNA

SEQ ID NO: 50 - protein, 362 aa, Unknown
  tree shrew
KISQYACQRRTTLNNCNRVFTDAFETLAENYEFRENEDSSVIFLRAASVLRSLPFTITSMRDTEGLPCLGDKVKCVIEEIIEDGESSEVNAVLNDERYKSFKLFTSVFGVGLKTSEKWFRMGFRTLSRVRSDKSLHLTRMQQAGFLYYEDLASCVTRAEAEAVGVLVKEAVGAFLPDALVTITGGFRRGKKTGHDVDFLITSPGSTEEKEEELLQKVLNLWEKKGLLLYYDLVESTFEKLKTPSRKVDALDHFPKCFLILKLHHQRVDGDKPSQQEGKSWKAIRVDLVMCPYERHAFALLGWTGSRQFERDLRRYATHERKMMLDNHALYDKTKRVFLKAESEEDIFAHLGLDYIEPWERNA

SEQ ID NO: 51 - protein, 375 aa, Ornithorhynchus anatinus
QVSQYACERRTTLNNCNQKFTDAFEILAKDFEFRENEGICLAFMRAISVLKCLPFTIVRMKDIEGVPWLGDQVKSIIEEIIEDGESSSVKAVLNDERYRSFQLFNSVFEVGLTDNGENGIARGFQTLNEVITDENISLTKTTLSTSLWNYLPGFLYYEDLVSCVAKEEADAVYLIVKEAVRAFLPEALVTLTGGFRRGKKIGHDVDFLISDPESGQDEQLLPNIIKLWEKQELLLYYDLVESTFEKTKIPSRKVDAMDHFQKCFLILKLHHQKVDSGRYKPPPESKNHEAKNWKAIRVDLVMCPFEQYAYALLGWTGSRQFERDLRRYATHEKKMMLDNHALYDKTKKIFLKAESEEDIFTHLGLDYIEPWERNA

SEQ ID NO: 52 - protein, 365 aa, Serinus canaria
KVSQYSCQRKTTLNNYNKKFTDAFEVMAENYEFKENEIFCLEFLRAASLLKSLPFSVTRMKDIQGLPCMGDQVRDVIEIIEEGESSRVKEVLNDERYKAFKQFTSVFGVGVKTSEKWYRMGLRTVGEVKADKTLKLSKMQKAGFLYYEDLVSCVSKAEADAVSLIVKNTVCTFLPDALVTITGGFRRGKNIGHDIDFLITNPGPREDDELLHKVIDLWKKQGLLLYCDIIESTFVKEQLPSRKIDAMDHFQKCFAILKLYQPRVDNSTCNTSKKLEMAEVKDWKAIRVDLVITPFEQYSYALLGWTGSRQFGRDLRRYAAHERRMILDNHGLYDRTKRIFLKAGSEEEIFAHLGLDYVEPWERNA

SEQ ID NO: 53 - protein, 366 aa, Neopelma chrysocephalum
KVSQYSCQRKTTLNNYNKKFTDAFEIMAENYEFKENEIFCLEFLRAASLLKYLPFPVTRMKDIQGLPCIGDQVRDVIEGIIEEGESSRVKEVLNDERYKAFKQFTSVFGVGVKTSEKWYRMGLRTVEELKADKTLKLSKMQKAGFLYYEDLVSCVSKAEADAVTLIVKNTVSTFLPDALVTITGGFRRGKKMGHDIDFLITNPGPREDDELLHKVVDLWKKQGLLLYCDIIESTFVEEQLPSRKVDAMDNFQKCFTILKLYQPGVDNSSYNMSKKSDMAEVKDWKAIRVDLVITPFEQYAYALLGWTGSREFGRDLRRYASHERKMILDNHGLYDRRKRIFLKAGSEEEIFAHLGLDYVEPWERNA

SEQ ID NO: 54 - protein, 366 aa, Alligator sinensis
KVSQYACQRRTTLNNYNKKFTDAFEILAENCEFRENRLGCLEFLRAASVLKFLPFPIVKMKNIEGLPCMGDKVKCVIEEILEEGESCQAKEILNDERYKSFKLFTSVFGVGLKTTEKWYRMGFRTLEEVKAEKTLKLSRMQIAGFLHYEDIISYVSKAEADAVSLLIKDTVCMFLPDALVTITGGFRRGKKTGHDVDFLITNPGPEEEKELLHKVVDLWEKQGLLLYYDVIESTFEKEKRPSRKVDALDHFQKCFAILKLHQQRRGNSNSNISKESDKAEVKDWKAIRVDLVISPFEQYAYALLGWTGSRQFERDLRRYASRERKMMLDNHALYDKTKRTFLKAESEEEIFAHLGLDYIEPWERNA

SEQ ID NO: 55 - protein, 366 aa, Alligator sinensis
KISQYACQRRTTLNNYNKKFTDAFEILAENYEFNENKGFCLAFRRAASVLKFLPFTIVRMNDIQGLPWMGDQVKRVIEEILEEGESSKVKEVLNDEKYKAFKLFTSVFGVGRKTSEKWYRMGLRTLEEVKADKTLKLTKMQKAGFLYYEDLISRVSKAEADAVSLIVKDTVWKFLPDALVTLTGGFRLGKKMGHDVDFLITTPGPEEEEELLHKVIDLWKKQGLLLYYDIIESTFEKTKLPSRTVDALDHFQKCFAILKLHKQRVDNGNSNQSKEFDKEETKDWKAIRVDLVITPFEQYAYALLGWTGSAFFNRDLRRYATHEKKMMLDNHALYDKTKRIFLKAASEEEIFAHLGLDYLEPWERNA

SEQ ID NO: 56 - protein, 363 aa, Xenopus laevis
KVSQYACQRCTTLQDTNRIFTDAFDILAEHFEFCENKGRTVAFLRASSLIKSLPFPITAMKELEGLPWLGDQMKGIIEEILEEGKSYKVLEVMNEERYKSFKQFTSVFGVGLKTSDKWFRMGFRTLEEIKNEKELKLTKMQKCGLLYYEDITSYVSRAEAETTEQLIKSIVWKFVPDAIVTLTGGFRRGKKKGHDVDILITCARKGKEKNILHNTMSVLKNRGLLLFYNIIESTFDETKLPSRHVDALDHFQKCFTILKLPKRQMDIGNIIDPHECERKNWKAVRLDLVITPYEQYPYALLGWTGSRQFERDLRRYATHEKRMMLDNHGLYDKTKNNFLKANNEEDIFKQLGLDYLEPWERNA

SEQ ID NO: 57 - protein, 363 aa, Xenopus laevis
KVSQYACQRRTTLQDTNRIFTDAFDILAENYEFNENKGPCVAFRRASSVLKSLPFPIVAMKDLEGLPWLGDQMKRIIEEILEEGKSSKVVEVMNDERYKAFKLFTSVFGVGRKTSEKWFRMGFRTLEEIKSDKELKLTKMQKAGLLYYEDITSAVSKAEAEAVEQLIKSIVWKFVPDAIVTLTGGFRLGKKMGHDVDILITTPRKGEKEEILHKTINVLKKRGLLLFYNIIESTFDETKLPSRHVDALDHFQKCFTILKLQKQQLDNGNSNEPFETEKKNWKAIRVDLVITPYEQYAYALLGWTGSPQFNRDLRRYATHEKRMMLDNHALYDKTKNIFLKANSEEDIFTHLGLDYLEPWERNA

SEQ ID NO: 58 - protein, 367 aa, Unknown
  Notechis
SISQYACQRRTTLNNYNKKFTDAFEILAENYEFNENKGFCLAFRRAASVLKFLPFTIVRVNDIEGLPWMGEQVKRIIEDILEEGESSKVKAVLNNENYRAFKLFTSVFGVGRKTSEKWYRMGLRTLEEVKADKNLKLTRMQKAGFLHYEDLISRVSKAEADAASLIVKDTVWKFLPNALVTLTGGFRLGKKMGHDVDFLITVPGSRQEEEELLHTVIDLWKKQGLLLYYDLIESTFEKTKLPSRTVDALDHFQKCFAILKLHKERVDKGNSIQSKAFAEEETKDWKAIRVDLVVTPFEQYAFALLGWTGSPFFNRDLRRYATHEKKMMLDNHALYDKTKKIFLKAASEEEIFAHLGLDYLEPWERNA

SEQ ID NO: 59 - protein, 363 aa, Salmo trutta
NVSQYACLRRTTTENHNKIFTDVLEELAENSEFNESKGPCLAFRRAASVLKSLPSAVHCLGAIQGLPCLGEHTKAVMEEILIFGRSFKVEEVQSDERYQALKLFTSVFGVGPKTAEKWYRRGLRSLKEILAEPNIQLNRMQRAGFLYYRDISKAVSKAEAKALSSIIEETAHWIAPDSILALTGGFRRGKEYGHDVDFLLTMPEMGKEEGLLLRVIDRLRDQGILLYCEHQDSTFDMSKLPSHRFEAMDHFEKCFLILRLEEGQVEGDGGLQKDPGESRGWRAVRVDLVAPPVDRYAFVLLGWTGSRQFERDLRRFASKERGMCLDNHALYDKTKKLFLPATSEEDIFAHLGLEYVEPWQRNA

SEQ ID NO: 60 - protein, 363 aa, Salmo trutta
TVSQYACQRRTTMENHNKIFTDAFEVLAENSEFNESKESRDAFRRAASVLKSLPSAVRSLGDTEGLPCLGEHTKRVIEEILEYGRSSKVEDILSDERYQTMKLFTSVFGVGPKTAEKWYRRGLRSLEEVRAEPSIHLNRMQRAGFLYYRDISKRVSKAEAKALGNIIEETVHAIAPDAIVTLTGGFRLGKEFGHDVDFILTTPEMGKEEGLLPRVIDRLRNQGILLYCDYQESTFDMSKLPSRTFEAMDHFQKCFLIIKLKEGLVEGEGGLQSDPGDRRGWRAVRVDLVAPPVDRYAFALLGWTGSPQFNRDLRRFARLERRMLLDNHALYDKTKKIFLPAKTEEDIFAHLGLEYIEPWQRNA

SEQ ID NO: 61 - protein, 360 aa, Electrophorus electricus
TVSQYACQRRTTLDNHNKVFTDALEVLIENYEFSDNKGACVGFRRAASVLKSLPKPLRCLKDMEGLPCLGDDTKAIIEEIYECGSSSRVENILSDEKYQTLKLFTSVFGVGPKTGEKWYRRGLRALEQVHSEPSIQLNKMQAAGFLYYEDISKPVSRAEAKAVGCIIEEVASCFSSSVTITLTGGFRRGKEFGHDVDFLLSIPEPGKEDGLLPAVIDRLRKQGILLYSDLQESTLQQWKRPSRCFDSMDHFQKCFLIVKLWTRLVEGHREDPSSQRDWKAVRVDLVVPPVDCYAFALLGWSGSTQFERDLRRFARLERRMLLDNHALYDKTTNTFLQAKTEEDIFAHLGLDYIEPWQRNA

SEQ ID NO: 62 - protein, 360 aa, Electrophorus electricus
TVSQYACQRRTTLDNHNKIFTDALEVLAENYEFSDSKESRDAFRRAASVLKSLPTPLRSLQDTEGLPCLGEETKRVIEEIFEEGSSSRVEDILSDERYQTLKLFTSVFGVGPKTAEKWYRRGLRSLEQVRSDPSIHLNRMQTAGFLYYEDISKPVSKAEAKALGNIIEETASAFSPSVTITLTGGFRLGKEFGHDVDFLLTVPEPGKEDGLLPAVIDRLRSQGILLYSDFQESTFDLSKLPSRRFEAMDHFQKCFLIVKLKAGLVEGEGADPGDRRDWRAVRVDLVAPPVDRYAFALLGWSGSAFFNRDLRRFARLERGMLLDNHALYDKTTNTFLQAKTEEDIFAHLGLDYIEPWQRNA

SEQ ID NO: 63 - protein, 361 aa, Anabas testudineus
TVSQYACQRRTTTENNNKILTDAFEVLAESYELNQLEGPCLAFRRAASVLKSLPWAVQCLGATQGLPCLGEHTKALIEEILQYGHSFEVEKILSDERYQTLKLFTSVFGVGPKTAEKWYRRGLRSFSDILAEPSIQLNRMQQSGFLHYGDISRAVSKAEARALGNIIDEAVHAITPDGILALTGGFRRGKEFGHDVDFIVTTPEQGKEETLLPNIIDRLKEQGILLYSDYQTSTFDISKLPSHKFEAMDHFAKCFLILRLEGSLVDRGLNSTEGDSRGWRAVRVDLVSPPMERYAYALLGWTGSRQFERDLRRFARLEQHMLLDNHALYDKTKKEFLAATTERDIFAHLGLEYIEPWQRNA

SEQ ID NO: 64 - protein, 361 aa, Anabas testudineus
TVSQYACQRRTTTENNNKIFTDAFEVLAESYEFNEMEESRDAFRRAASVLKSLPWAVQSLGATQDLPCLGEHTKRVMEEILQYGRSFEVEKILSDERYQTLKLFTSVFGVGPKTAEKWYRRGLRSFEDIRAEPSIHLNRMQQSGFLHYGDISRAVSKAEARALGNIIDEAVHAITPDAILALTGGFRLGKEFGHDVDFIVTTPELGKEESLLPNIIDRLKKQGILLYSDYQASTFDMSKLPSHRFEAMDHFAKCFLILRLEGSQVEGGLNSAEGDSRGWRAVRVDLVSPPMDRYAFALLGWTGSAFFNRDLRRFARMERRMLLDNHALYDKTKKEFLAATTEKDIFAHLGLEYIEPWQRNA

SEQ ID NO: 65 - protein, 361 aa, Poecilia reticulate
KVSQYACQRRTTVENNNRIFTDAFEVLAENYEFNEIEGRCLAFRRAASVLKSLPWAVRSVGATLDLPCLGEHTTAVMKEILQYGRSFEVEKILSDERCQTLKLFTSVFGVGPKTAEKWYRRGLRSFSDVLAQPGIHLNRMQQSGFLHYGDISRAVSKAEARAVGNIIDEAVHVITPNAILALTGGFRRGKDFGHDVDFIVTTTELGKEKNLLISVIESLKKQGLLLFSDYQASTFDISKLPSHRFEAMDHFAKCFLILRLEGSRVEGGLQRAQADGRGWRAVRVDLVSPPADRFAFTMLGWTGSRMFERDLRRFARLERQMLLDNHALYDKTKKEFLTAATEKDIFDHLGLEYIEPWQRNA

SEQ ID NO: 66 - protein, 366 aa, Artificial Sequence; identical to SEQ ID NO: 55
  F57 trunc new (Poecilia reticulate) guppy

SEQ ID NO: 67 - protein, 362 aa, Rattus norvegicus
KISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 68 - protein, 366 aa, Artificial Sequence
  N100 trunc new (short Rattus norvegicus)
SVVQKISEYACQRRTTLNNHNEVFTRAFDILAENYEFRENEESRDAFRRASSVLKSLPFPIISMKDTEGIPCLGDKVKRVIEEIIEDGESSEVKAVLNDERYKAFKLFTSVFGVGRKTAEKWFRMGFRTLEKIKSDKSLRFTQMQKAGFLYYEDLAERVNRAEAEAIEMLVKEAVVAFLPDALVTMTGGFRLGKMTGHDVDFLITSPEATEEEEQQLLHKVTDFWRQQGLLLYCDIIESTFEKFKLPSRTVDALDHFQKCFLILKLHHGRVVSEKSGQQEGKGWKAIRVDLVMCPYERRAFALLGWTGSPFFNRDLRRYATHERKMMLDNHALYDKTKRVFLEAESEEEIFAHLGLDYIEPWERNA

SEQ ID NO: 69 - protein, 362 aa, Piliocolobus tephrosceles
KISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSALVKEAVWAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSDQSSWQGGKTWKAIRVDLVMCPYERRAFALLGWTGSRQFERDLRRYATHERKMILDNHALYDKTKRIFLKAESEEEIFAHLGLDYIEPWERNA

SEQ ID NO: 70 - protein, 362 aa, Sus scrofa
KISQYACQRRTTLNNCNHIFTDAFEVLAENYEFRENETFCLAFMRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDKTKRIFLKAESEEEIFAHLGLDYLEPWERNA

SEQ ID NO: 71 - protein, 362 aa, Artificial Sequence
  N27 trunc new  [88.13 percent identity to wt calc by NCBI BLASTP] pig
KISQYACQRRTTLNNYNEIFTRAFEILAENYEFRENEESYVTFRRAASVLKSLPFTIISMKDTEGIPCLGDKVKRVIEEIIEDGESSEVKAVLNDERYKAFKLFTSVFGVGRKTSEKWFRMGFRTLEKIRSDKTLKFTRMQKAGFLYYEDLVSRVTRAEAEAVSVLVKEAVWAFLPDAFVTMTGGFRLGKKIGHDVDFLITSPGSTEEEEQQLLHKVINLWEKKGLLLYYDLVESTFEKLKLPSRTVDALDHFQKCFLILKLHHQRVGSGKSSQQEGKTWKAIRVDLVMCPYERRAFALLGWTGSPQFNRDLRRYATHERKMMLDNHALYDKTKRIFLKAESEEEIFAHLGLDYIEPWERNA

SEQ ID NO: 72 - protein, 362 aa, Artificial Sequence
  N35 trunc new
KISQYACQRRTTLNNYNEIFTRAFEVLAENYEFRENEESYVTFRRAASVLKSLPFTIISMKDTEGIPCLGDKVKRVIEEIIEDGESSEVKAVLNDERYQAFKLFTSVFGVGRKTSEKWFRMGFRTLEKIRSDKTLKFTRMQKAGFLYYEDLAERVTRAEAEAVSVLVKEAVWAFLPDAFVTMTGGFRLGKKIGHDVDFLITSPGSTEEEEQQLLPKVINLWERKGLLLYYDLVESTFEKLKLPSRKVDALDHFQKCFLILKLHHQRVGSGKSSQQEGKTWKAIRVDLVMCPYERRAFALLGWTGSAFFNRDLRRYATHERKMILDNHALYDKTKRIFLKAESEEEIFAHLGLDYIEPWERNA

SEQ ID NO: 73 - protein, 361 aa, Bubalus bubalis
KISQYACQRKTTLNNYNHIFTDAFEILAENSEFKENEVSYVTFMRAASVLKSLPFTIISMKDTQGIPCLGDKVKCVIEEIIEDGESSEVKAVLNDERYQSFKLFTSVFGVGLKTSEKWFRMGFRSLSKITSDKTLKFTKMQKAGFLYYEDLVSCVTRAEAEAVGVLVKEAVWAFLPDAFITMTGGFRRGKKIGHDVDFLITSPGSAEDEEQLLPKVINLWEKKGLLLYYDLVESTFEKFKLPSRQVDTLDHFQKCFLILKLHHQRVDSGRSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 74 - protein, 362 aa, Marmota flaviventris
TISQYACQRRTTLNNCNRVFTDAFDVLAENYEFRENESCSVVFMRAASVLKSLPFTIISMRDLEGIPCLEGKAKSIIEEIIEDGESSEVKAVLNDERYKSFKLFTSVFGVGLKTSEKWFRMGFRTLSKIRSDKSLKFTHMQKAGFLYYEDLVSCVTRAEAEAVSVLVKEAVWAFLPDAFITMTGGFRRGKNIGHDVDFLITSAEATEEEEQQLLHKVTNLWEKKGLLLYCDLVESTFEKLKTPSRKVDALDHFQKCFLILKLHHQRVDSDKASQQGGKNWKAIRVDLVMCPYERRAFALLGWTGSRQFERDLRRYATHERKMILDNHALYDKTKRIFLKAESEEEIFAHLGLDYIEPWERNA

SEQ ID NO: 75 - protein, 362 aa, Artificial Sequence
  Truncated mouse TdT M53
KISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLAERVNRPEAEAIRMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 76 - protein, 379 aa, Artificial Sequence
  N93  [95.15 percent identity to wt (Marmota flaviventris)calc by NCBI BLASTP]
SPSPVPGSQNVPAPAVQTISQYACQRRTTLNNYNRVFTDAFDVLAENYEFRENEESSVVFRRAASVLKSLPFTIISMRDLEGIPCLEGKAKRIIEEIIEDGESSEVKAVLNDERYKAFKLFTSVFGVGRKTSEKWFRMGFRTLEKIRSDKSLKFTHMQKAGFLYYEDLVSRVTRAEAEAVSVLVKEAVWAFLPDAFITMTGGFRLGKNIGHDVDFLITSAEATEEEEQQLLHKVTNLWEKKGLLLYCDLVESTFEKLKLPSRTVDALDHFQKCFLILKLHHQRVDSDKASQQGGKNWKAIRVDLVMCPYERRAFALLGWTGSPQFNRDLRRYATHERKMILDNHALYDKTKRIFLKAESEEEIFAHLGLDYIEPRERNA

SEQ ID NO: 77 - protein, 366 aa, Artificial Sequence
  N72 [91.76 percent identity to wt(Bubalus bubalis) calc by NCBI BLASTP]
SVVKKISQYACQRRTTLNNYNEIFTRAFEILAENYEFKENEESYVTFRRAASVLKSLPFTIISMKDTEGIPCLGDKVKRVIEEIIEDGESSEVKAVLNDERYQAFKLFTSVFGVGRKTSEKWFRMGFRSLEKIRSDKTLKFTRMQKAGFLYYEDLVSRVTRAEAEAVGVLVKEAVWAFLPDAFVTMTGGFRLGKKIGHDVDFLITSPGSTEDEEQQLLPKVINLWERKGLLLYYDLVESTFEKFKLPSRQVDALDHFQKCFLILKLHHQRVGSGKSSQQEGKTWKAIRVDLVMCPYEQRAFALLGWTGSAFFNRDLRRYATHERKMMLDNHALYDKTKRIFLKAESEEEIFAHLGLDYIEPRERNA

SEQ ID NO: 78 - protein, 379 aa, Artificial Sequence
  N35  trunc new [89.18 percent identity to wt (Panthera tigris altaica) calc by NCBI BLASTP]
SPSPVPGSQNVPAPVVKKISQYACQRRTTLNNYNEIFTRAFEVLAENYEFRENEESYVTFRRAASVLKSLPFTIISMKDTEGIPCLGDKVKRVIEEIIEDGESSEVKAVLNDERYQAFKLFTSVFGVGRKTSEKWFRMGFRTLEKIRSDKTLKFTRMQKAGFLYYEDLAERVTRAEAEAVSVLVKEAVWAFLPDAFVTMTGGFRLGKKIGHDVDFLITSPGSTEEEEQQLLPKVINLWERKGLLLYYDLVESTFEKLKLPSRKVDALDHFQKCFLILKLHHQRVGSGKSSQQEGKTWKAIRVDLVMCPYERRAFALLGWTGSAFFNRDLRRYATHERKMILDNHALYDKTKRIFLKAESEEEIFAHLGLDYIEPWERNA

SEQ ID NO: 79 - protein, 379 aa, Artificial Sequence
  N27 trunc new  [88.13 percent identity to wt(Sus scrofa) calc by NCBI BLASTP]
SPSPVPGSQNVPAPVVKKISQYACQRRTTLNNYNEIFTRAFEILAENYEFRENEESYVTFRRAASVLKSLPFTIISMKDTEGIPCLGDKVKRVIEEIIEDGESSEVKAVLNDERYKAFKLFTSVFGVGRKTSEKWFRMGFRTLEKIRSDKTLKFTRMQKAGFLYYEDLVSRVTRAEAEAVSVLVKEAVWAFLPDAFVTMTGGFRLGKKIGHDVDFLITSPGSTEEEEQQLLHKVINLWEKKGLLLYYDLVESTFEKLKLPSRTVDALDHFQKCFLILKLHHQRVGSGKSSQQEGKTWKAIRVDLVMCPYERRAFALLGWTGSPQFNRDLRRYATHERKMMLDNHALYDKTKRIFLKAESEEEIFAHLGLDYIEPWERNA

SEQ ID NO: 80 - protein, 379 aa, Artificial Sequence
  N14 trunc new  [92.08 percent identity to wt(Piliocolobus tephrosceles) calc by NCBI BLASTP]
SPSPVPGSQNVPAPVVQKISQYACQRRTTLNNYNEIFTRAFDILAENSEFRENEDSYVTFRRAASVLKSLPFTIISMKDTEGIPCLGSKVKRIIEEIIEDGESSEVKAVLNDERYQAFKLFTSVFGVGRKTSEKWFRMGFRTLEKVRSDESLKFTRMQRAGFLYYEDLVSRVTRAEAEAVSVLVKEAVWAFLPDAFVTMTGGFRLGKKMGHDVDFLITSPGSTEDEEQQLLQKVMNLWEKKGLLLYYDLVESTFEKLRLPSRTVDALDHFQKCFLIFKLPLQRVGSDQSSWQEGKTWKAIRVDLVMCPYERRAFALLGWTGSAFFNRDLRRYATHERKMILDNHALYDKTKRIFLKAESEEEIFAHLGLDYIEPWERNA

SEQ ID NO: 81 - protein, 379 aa, Artificial Sequence
  N100 or trunc new  [93.14 percent identity to wt(long Rattus norvegicus) calc by NCBI BLASTP]
SPSPVPGSQNVPAPVVQKISEYACQRRTTLNNHNELFTRAFDILAENYEFRENEESRDAFRRAASVLKSLSFPIVSMKDIEGIPCLGDKVKRIIEEIIEDGESSEVKAVLNDERYKAFKLFTSVFGVGRKTAEKWFRMGFRTLEKIRSDKSLRFTHMQKAGFLYYEDLVSRVNRAEAEAVSMLVKEAVVAFLPDALVTMTGGFRLGKMTGHDVDFLITSPEATEEEEQQLLHKVTNFWRQQGLLLYCDIIESTFEKFKLPSRTVDALDHFQKCFLILKLHRGLVRSEESGQQEGKDWKAIRVDLVMCPYERRAFALLGWTGSPFFNRDLRRYATHERKMMLDNHALYDKTKRVFLEAESEEEIFAHLGLDYIEPWERNA

SEQ ID NO: 82 - protein, 366 aa, Artificial Sequence; identical to SEQ ID NO: 68
  N100 trunc new  [89.86 percent identity to wt short Rattus norvegicus calc by NCBI BLASTP]

SEQ ID NO: 83 - protein, 383 aa, Artificial Sequence
  F57 trunc new (Poecilia reticulate)  [56.66 percent identity to wt calc by NCBI BLASTP]
SPSPVPGSQNVPAPSVDKISQYACQRRTTLNNYNKKFTDAFEILAENYEFNENKGFCLAFRRAASVLKFLPFTIVRMNDIQGLPWMGDQVKRVIEEILEEGESSKVKEVLNDEKYKAFKLFTSVFGVGRKTSEKWYRMGLRTLEEVKADKTLKLTKMQKAGFLYYEDLISRVSKAEADAVSLIVKDTVWKFLPDALVTLTGGFRLGKKMGHDVDFLITTPGPEEEEELLHKVIDLWKKQGLLLYYDIIESTFEKTKLPSRTVDALDHFQKCFAILKLHKQRVDNGNSNQSKEFDKEETKDWKAIRVDLVITPFEQYAYALLGWTGSAFFNRDLRRYATHEKKMMLDNHALYDKTKRIFLKAASEEEIFAHLGLDYLEPWERNA

SEQ ID NO: 84 - protein, 378 aa, Artificial Sequence
  F32 trunc new  [89.95 percent identity to wt Anabas testudineus calc by NCBI BLASTP]
SPSPVPGSQNVPAPSVATVSQYACQRRTTTENNNKIFTDAFEVLAESYEFNEMEESRDAFRRAASVLKSLPWAVQSLGATQDLPCLGEHTKRVMEEILQYGRSFEVEKILSDERYQTLKLFTSVFGVGPKTAEKWYRRGLRSFEDIRAEPSIHLNRMQQSGFLHYGDISRAVSKAEARALGNIIDEAVHAITPDAILALTGGFRLGKEFGHDVDFIVTTPELGKEESLLPNIIDRLKKQGILLYSDYQASTFDMSKLPSHRFEAMDHFAKCFLILRLEGSQVEGGLNSAEGDSRGWRAVRVDLVSPPMDRYAFALLGWTGSAFFNRDLRRFARMERRMLLDNHALYDKTKKEFLAATTEKDIFAHLGLEYIEPWQRNA

SEQ ID NO: 85 - protein, 377 aa, Artificial Sequence
  F24 trunc new  [83.55 percent identity to wt Electric eel calc by NCBI BLASTP]
SPSPVPGSQNVPAPSLPTVSQYACQRRTTLDNHNKIFTDALEVLAENYEFSDSKESRDAFRRAASVLKSLPTPLRSLQDTEGLPCLGEETKRVIEEIFEEGSSSRVEDILSDERYQTLKLFTSVFGVGPKTAEKWYRRGLRSLEQVRSDPSIHLNRMQTAGFLYYEDISKPVSKAEAKALGNIIEETASAFSPSVTITLTGGFRLGKEFGHDVDFLLTVPEPGKEDGLLPAVIDRLRSQGILLYSDFQESTFDLSKLPSRRFEAMDHFQKCFLIVKLKAGLVEGEGADPGDRRDWRAVRVDLVAPPVDRYAFALLGWSGSAFFNRDLRRFARLERGMLLDNHALYDKTTNTFLQAKTEEDIFAHLGLDYIEPWQRNA

SEQ ID NO: 86 - protein, 367 aa, Artificial Sequence
  F15 trunc new  [80.38 percent identity to wt Salmo truttal calc by NCBI BLASTP]
SSVATVSQYACQRRTTMENHNKIFTDAFEVLAENSEFNESKESRDAFRRAASVLKSLPSAVRSLGDTEGLPCLGEHTKRVIEEILEYGRSSKVEDILSDERYQTMKLFTSVFGVGPKTAEKWYRRGLRSLEEVRAEPSIHLNRMQRAGFLYYRDISKRVSKAEAKALGNIIEETVHAIAPDAIVTLTGGFRLGKEFGHDVDFILTTPEMGKEEGLLPRVIDRLRNQGILLYCDYQESTFDMSKLPSRTFEAMDHFQKCFLIIKLKEGLVEGEGGLQSDPGDRRGWRAVRVDLVAPPVDRYAFALLGWTGSPQFNRDLRRFARLERRMLLDNHALYDKTKKIFLPAKTEEDIFAHLGLEYIEPWQRNA

SEQ ID NO: 87 - protein, 384 aa, Artificial Sequence
  B63 trunc new  [85.68 percent identity to wt Notechis scutatus calc by NCBI BLASTP]
SPSPVPGSQNVPAPSVDSISQYACQRRTTLNNYNKKFTDAFEILAENYEFNENKGFCLAFRRAASVLKFLPFTIVRVNDIEGLPWMGEQVKRIIEDILEEGESSKVKAVLNNENYRAFKLFTSVFGVGRKTSEKWYRMGLRTLEEVKADKNLKLTRMQKAGFLHYEDLISRVSKAEADAASLIVKDTVWKFLPNALVTLTGGFRLGKKMGHDVDFLITVPGSRQEEEELLHTVIDLWKKQGLLLYYDLIESTFEKTKLPSRTVDALDHFQKCFAILKLHKERVDKGNSIQSKAFAEEETKDWKAIRVDLVVTPFEQYAFALLGWTGSPFFNRDLRRYATHEKKMMLDNHALYDKTKKIFLKAASEEEIFAHLGLDYLEPWERNA

SEQ ID NO: 88 - protein, 380 aa, Artificial Sequence
  B59 trunc new [84.74 percent identity to wt xenopus calc by NCBI BLASTP]
SPSPVPGSQNVPAPSEVKVSQYACQRRTTLQDTNRIFTDAFDILAENYEFNENKGPCVAFRRASSVLKSLPFPIVAMKDLEGLPWLGDQMKRIIEEILEEGKSSKVVEVMNDERYKAFKLFTSVFGVGRKTSEKWFRMGFRTLEEIKSDKELKLTKMQKAGLLYYEDITSAVSKAEAEAVEQLIKSIVWKFVPDAIVTLTGGFRLGKKMGHDVDILITTPRKGEKEEILHKTINVLKKRGLLLFYNIIESTFDETKLPSRHVDALDHFQKCFTILKLQKQQLDNGNSNEPFETEKKNWKAIRVDLVITPYEQYAYALLGWTGSPQFNRDLRRYATHEKRMMLDNHALYDKTKNIFLKANSEEDIFTHLGLDYLEPWERNA

SEQ ID NO: 89 - protein, 383 aa, Artificial Sequence; identical to SEQ ID NO: 83
  B57 trunc new [83.03 percent identity to wt alligator calc by NCBI BLASTP; 84.07 % id to purple]

SEQ ID NO: 90 - protein, 370 aa, Artificial Sequence
  B19 trunc new [92.35 percent identity to wt neopelma calc by NCBI BLASTP]
SSVSKVSQYSCQRKTTLNNYNKKFTDAFEIMAENYEFKENEIFCLEFRRAASVLKFLPFPVTRMKDIQGLPCMGDRVRDVIEEIIEEGESSRVKEVLNDERYKAFKQFTSVFGVGRKTSEKWYRMGLRTLEEVKADKTLKLSKMQKAGFLYYEDLAERVSKAEADAVSLIVKNTVVTFLPDALVTITGGFRLGKKIGHDIDFLITNPGPREDEELLHKVVDLWKKQGLLLYCDIIESTFVKEQLPSRKVDAMDNFQKCFAILKLYQPRVDNSSYNTSKKFDMAEVKDWKAIRVDLVITPFEQYAYALLGWTGSPQFNRDLRRYATHERKMILDNHALYDRRKRIFLKAGSEEEIFAHLGLDYVEPRERNA

SEQ ID NO: 91 - protein, 369 aa, Artificial Sequence
  specific bovine from Misha
KTPPLVVKKISQYACQRKTTLNNYNEIFTRAFEILAENSEFKENEESYVTFRRAASVLKSLPFTIISMKDTEGIPCLGDKVKRIIEEIIEDGESSEVKAVLNDERYQAFKLFTSVFGVGRKTSEKWFRMGFRSLEKIRSDKTLKFTKMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSAFFNRDIRRYATHERKMMLDNHALYDKTKRVFLKAESEEEIFAHLGLDYIEPRERNA

SEQ ID NO: 92 - protein, 374 aa, Artificial Sequence
  specific latmeria from Misha
NVPAPSVVAISQYACQRRTTLNNHNKIFTDAFEILAENYEFNENEGPCLAFRRAASLLKSLPYAISSMKDLEGLPCLGDQTKAVIEEILEEGQSSKVQDVLSDERYKSIKLFTSVFGVGLKTAEKWYRKGFRTLEEVQADKEIKLTKMQKAGFLYYEDISSAVTKAEAEAIGQIIEDTVRLFAPDAIVTLTGGFRLGKKIGHDVDFLITTPETGNENGLLHKVINVLQNQGILLYYDIVESTFDKTRLPSRKVDALDHFQKCFAILKLHKQKVNTSNSEEAEEPSNTETKDWKAIRVDLVITPFDQYAFALLGWTGSAFFNRDLRRFATHERKMMLDNHALYDKTKKIFLPAKTEEDIFAHLGLDYIEPWERNA

SEQ ID NO: 93 - protein, 370 aa, Artificial Sequence
  specific Puma from Misha
KTESAVVKKIPLYACQRRTTLNNFNEIFTRAFEVLAENYEFKENEISSATFRRAASVLKSLPFTIISMKDTEGIPCLGDKVKRVIEEIIEDGESSEVKAVLNDERYQAFKLFTSVFGVGLKTSEKWFRMGFRTLEKIKSDKTLKFTQMQKAGFLYYEDLVSGVTRAEAEAVGVLVKEAVWAFLPDAFVTMTGGFRLGKKIGHDVDFLITIPGSTDEEEEQLLPKVINLWQRKELLLYYDLVESTFEKLKLPSRKVDALDHFQKCFLILKLHHQRVDSGKCSQQEGKTWKAIRVDLVMCPYERRAFALLGWTGSAFFNRDLRRYATHERKMILDNHALYDKTKKIFLKAESEEEIFAHLGLDYIEPWERNA

SEQ ID NO: 94 - protein, 370 aa, Artificial Sequence; identical to SEQ ID NO: 93
  specific N139 from Misha

SEQ ID NO: 95 - protein, 370 aa, Artificial Sequence
  specific shrew from Misha
NVPAPVVQKISQYACQRRTTLNNHNHIFTDAFEILAENDEFRENEESRDAFRRAASVLKSLPFSIISMKDTEGIPCLADKVKRVIEEIIEDGESSEVKAVLNDERYKAFKLFTSVFGVGRKTAEKWFRLGFRTLEGIRNDKTLKLTHMQKAGFLYYEDLVSGVTRAEAEAVGVLVKEAVWAFLPDAIVTMTGGFRLGKKVGHDVDFLITSPEATEEQEQQLLHKVITFWEKEGLLLYCDLYESTFEKLKMPSRTVDALDHFQKCFLILKLHRESVDDGTSSQLQGKTWKAIRVDLVVCPYECRAFALLGWTGSPFFNRDLRRYATHERKMMLDNHALYDKTKRKFLSADSEEDIFAHLGLDYIEPRERNA

SEQ ID NO: 96 - protein, 381 aa, Artificial Sequence
  M27
NSSPSPVPGSQNVPAPVVKKISQYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 97 - protein, 383 aa, Artificial Sequence
  B10 trunc new [91.15 percent identity to wt canary calc by NCBI BLASTP]
SPSPVPGSQNVPAPSVTKVSQYSCQRKTTLNNYNKKFTDAFEVMAENYEFKENEESRDAFRRAASLLKSLPFPVTRMKDIQGLPCMGDQVRRIIEEIIEEGESSRVKEVLNDERYKAFKQFTSVFGVGRKTSEKWYRMGLRTVEEVKADKTLKLSKMQKAGFLYYEDLVSRVSKAEADAVSLIVKNTVVTFLPDALVTITGGFRLGKKIGHDIDFLITNPGPREDEELLHKVIDLWKKQGLLLYCDIIESTFVKEQLPSRKVDAMDHFQKCFAILKLYQPRVDNSTSNTSKKLEMAEVKDWKAIRVDLVITPFEQYAYALLGWTGSAFFNRDLRRYASHERKMILDNHALYDRRKRIFLKAGSEEEIFAHLGLDYVEPWERNA

SEQ ID NO: 98 - protein, 381 aa, Artificial Sequence
  Mouse TdT mutant  >TH M27
SGSPSPVPGSQNVPAPVVKKISQYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 99 - protein, 381 aa, Artificial Sequence
  Mouse TdT mutant >MS 13-34
SGSPSPVPGSQNVPAPVVKKISQYACQRRTTLNNYNELFTRALDILAENDEFRENEESRDAFRRASSVLKSLPFPITSMKDTEGIPCLGDKVKRIIEEIIEDGESSEVKAVLNDERYQAFKLFTSVFGVGRKTAEKWFRMGFRTLEKIRSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSAFFNRDLRRYATHERKMMLDNHALYDKTKRVFLEAESEEEIFAHLGLDYIEPRERNA

SEQ ID NO: 100 - protein, 381 aa, Artificial Sequence
  Mouse TdT mutant >MS 34-1
SGSPSPVPGSQNVPAPVVKKISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSAFFNRDLRRYATHERKMMLDNHALYDKTKRVFLEAESEEEIFAHLGLDYIEPWERNA

SEQ ID NO: 101 - protein, 381 aa, Artificial Sequence
  Mouse TdT mutant >TH c2_5
SGSPSPVPGSQNVPAPVVKKISQYACQRRTTLNNYNELFTRALDILAENDEFRENEESCLAFRRASSVLKSLPFPITSMKDTEGIPCLGDKVKRIIEEIIEDGESSEVKAVLNDERYKSFKLFTSVFGVGLKTAEKWFRMGFRTLEKIRSDKSLRFTQMQKAGFLYYEDLVSGVNRPEAEAVSTLVKEAVVTFLPDALVTMTGGFRLGHMTGHDVDFLITSPEATEDEEQQLLHKVTDFWKQQGLLLYCDILESTFEKFKQPSRKVDALDHFQKCFLILKLDHLRVHSAKSGQQEGKGWKAIRVDLVMCPYDRRAFALLGWTGSVQFKRDLRRYATHERKMMLDEHALYDKTKRVFLEAESEEEIFAHLGLDYIEPWERNA

SEQ ID NO: 102 - protein, 361 aa, Artificial Sequence
  (M57 without His tag & linker & with Q4E mutation) bovine-mouse chimera
KISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 103 - protein, 361 aa, Artificial Sequence
  (M59 without His Tag and linker & with Q4E mutation) bovine-mouse chimera
KISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLAERVNRPEAEAIRMLVKEAVVTFLPDALVTMTGGFRLGKMTGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 104 - protein, 362 aa, Artificial Sequence
  (M27 with maximal N-terminal deletion plus Q4E mutation) mouse
KISEYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 105 - protein, 361 aa, Artificial Sequence
  M54 with maximal N-terminal deletion plus Q4E mutation
KISEYACQRKTTLNNYNEIFTRAFEILAENSEFKENEESYVTFRRAASVLKSLPFTIISMKDTEGIPCLGDKVKRIIEEIIEDGESSEVKAVLNDERYQAFKLFTSVFGVGRKTSEKWFRMGFRSLEKIRSDKTLKFTKMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSAFFNRDIRRYATHERKMMLDNHALYDKTKRVFLKAESEEEIFAHLGLDYIEPRERNA

SEQ ID NO: 106 - protein, 362 aa, Artificial Sequence
  M77 without His tag
KKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 107 - protein, 361 aa, Artificial Sequence
  C1-40 without His tag
KISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 108 - protein, 363 aa, Artificial Sequence
  A2-20 without His tag
VTTVSEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 109 - protein, 363 aa, Artificial Sequence
  ii-ell-40 without His tag
VATVSEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 110 - protein, 363 aa, Artificial Sequence; identical to SEQ ID NO: 109
  9-40 without His tag

SEQ ID NO: 111 - protein, 365 aa, Artificial Sequence
  (45-751)(internal His tag)
MAEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTLEKFKLPSRQVDTLDHFQKCFLILKLHRQRVDSSKHHHHHHKTWKAIRVDLVMCPYENRAFALLGWTGSPQFNRDLRRYATHERKMMLDNHALYDKTKRVFLEAESEEEIFAHLGLDYIEPWERNA

SEQ ID NO: 112 - protein, 369 aa, Artificial Sequence
  (46-737) (internal His tag)
MAHHGSEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTLEKFKRPSRQVDTLDHFQKCFLILKLHRQRVDSSKHHHHHHETWKAIRVDLVMCPYENRAFALLGWTGSPQFNRDLRRYATHERKMMLDNHALYDKTKRVFLEAESEEEIFAHLGLDYIEPWERNA

SEQ ID NO: 113 - protein, 552 aa, Saccharomyces cerevisiae
MASQKVFGITGPVSTVGATAAENKLNDSLIQELKKEGSFETEQETANRVQVLKILQELAQRFVYEVSKKKNMSDGMARDAGGKIFTYGSYRLGVHGPGSDIDTLVVVPKHVTREDFFTVFDSLLRERKELDEIAPVPDAFVPIIKIKFSGISIDLICARLDQPQVPLSLTLSDKNLLRNLDEKDLRALNGTRVTDEILELVPKPNVFRIALRAIKLWAQRRAVYANIFGFPGGVAWAMLVARICQLYPNACSAVILNRFFIILSEWNWPQPVILKPIEDGPLQVRVWNPKIYAQDRSHRMPVITPAYPSMCATHNITESTKKVILQEFVRGVQITNDIFSNKKSWANLFEKNDFFFRYKFYLEITAYTRGSDEQHLKWSGLVESKVRLLVMKLEVLAGIKIAHPFTKPFESSYCCPTEDDYEMIQDKYGSHKTETALNALKLVTDENKEEESIKDAPKAYLSTMYIGLDFNIENKKEKVDIHIPCTEFVNLCRSFNEDYGDHKVFNLALRFVKGYDLPDEVFDENEKRPSKKSKRKNLSSGENLYFQGSSGS

SEQ ID NO: 114 - protein, 674 aa, Unknown
  Myceliophthora
MAERIYGVTPPISTALPTEEEKRLNNALHQELRAQGTFESPAETEKRKEVLRQLEKITNVFVQRAAAEKEPKNTILIRDAIGRVFTYGSYRLGVYGPGSDMDTLVVAPKYVTVEQYFRIFPEVLVEMAPPGAITDLTPVPEAFVPIIKFEFSGISIDLIFCSIQTLKQLPADKNWSLADNNLLRGLSENEVRSLNGTRVTDDILNLVPEPATFKLALRAIKLWAQRKAIYANIMGYPGGVAWAMLVARVCQLYPKATSAVIVNKFFHIMLKWPWPLPVLLKDIEYGCPVTRVAVWNPKIYASDRNHRMPIITPSYPSMCATHNVGRSSMAVIKDELEKGVQVTEDIMRGKRPWKDLFTKHTFFTSGFRYYLTVISSSRTKKAQNVWSGFVESRVRLLVNKLEMHPSISLARPFNKGYDREHRCKNNAQLEEVVSLGSLAYMYKPATGEAKAEAKKEVKTEVKHESGASVKNEAPEAKGEDGVRAKRENGDEAQLPPATGIKPEPTDGAGADVKLEDIPVKKDEPEEMTIYTTNHYIGLQLVEGAKTLDLSREVNDWKAMCTSNELYEEGTMFLSIQHVRNTALPDDVFEPGETRPRPAKKSLKRVASEDPGNKQTQPPAKKQVQDKAPPAAQQQQQQQHQQQQQPASTAAAAAGSSGENLYFQGSSGSHHHHHH

SEQ ID NO: 115 - protein, 672 aa, Unknown
  Thielavia
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRVTDEILHLVPEPATFKLALRAIKLWAQRKAIYANIMGYPGGVAWAMLVARVCQLYPKATSAVIVNKFFNIMLKWPWPLPVLLKDIEYNGPVTRVPVWNPKLYASDRNHKMPIITPAYPSMCATHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQPSSTATAAAGSSGENLYFQGSSGSHHHHHH

SEQ ID NO: 116 - protein, 566 aa, Pyronema omphalodes
MSAPPKQYGTTPPINTNPPTKEDMERNSELVQELKNQNCFEEKAESDKRVKVLEILQTLGEAFVKKACAAKGLPDHLVNNSGGKIFTFGSYRLGAYGPGSDIDTLLVVPAHIERADFFQHVPKMLSELNPPAEEVSPVPDAFVPIIKFELQSISIDLIFAKVPSVNSIPRDMTLENKDILKGCDEANLRGLNGVRLTDELLGLVPHPVNFRMALRAIKLWAKSRAIYANVMGFPGGIAWAMLVAKICQLYPMAVSAVIVCKFFTIYTKWKWPQPVLLKDILDEGGQGAGLRVWNPKIYASDRGHLMPVITPNYPCMCSTHNITKSTKAVILREMDRSLNIIHEIMDGKSPWSKLFAPHTFFTQDYRYYLRVISAARTKDEQLLWSGLVESKLRHLVSKLEMLDNIQLAHPFNKGFEYEVECNNEDEVMRVIRGENIDQSEANGTAVDSNGKSSKVKAYTTSFYVGLVLDTSKSKQFDISWACHEFYDICKTWNLYNDDMHSIHVVNTRNFELPDNVFKPGETKPTKKTKTIKRKIPNAAGKKRGADEISAPDSKRAHINEVPPNAN

SEQ ID NO: 117 - protein, 598 aa, Tilletia indica
MTKHLGVTPIVSDASPTAHEIALETQLVTELTTQNCFESPQDSRLREVVLGKADKLVKEFVYRAAIARGLPESAARELGGKIFTFGSYRLGVHGPGSDIDTLCVVPKHVQREDFFTIFEQMLRQREDVTDVAPVPEAYVPLIAVNFMGIAIDFLFARLSLSRIDDSLDLSDNTILKNMDDQDIRSVGGSRVTDEILRLVPNVETFHTALRAIKLWAQRRAIYKNMVGFPGGVAWAMLVARICQLYPNGNAAVIIERFFIIMFQWNWPQPVMLKHHIYEADPILKVWNPRLYAADRSHRMPIITPAYPGMCATHNITASTQAVLTLEFKRGIEIMSAMSASITNPALPQTTWATLFESRRFFEEYKYYLQIIASSGSADLQLKWAGTVESKLRHLVLSTEAQPGVKIAHPYTREIDLVSECHTDEEVRRVATGDVPPEVAARARTVEGEGVLPESKEVDKLKEIESKEEEARKKGRRTIWTTTFYIGLALHDGSDDKTGQSAPKRLDLVGPIQDFKGRCTEWATYDESTMGVVVRHVKRSQLPDSVFPDGKRPAAPVKKVVKKRKRNAGMAEAAAETKEEEGADEQEAKRARTNGDSAA

SEQ ID NO: 118 - protein, 574 aa, Clathrospora elynae
MNGPPKTQYGVTSAISEAAPTEEDRLLNDKLIETLKRENVFETPEGNAKREEVILHLQKVVEEFVRRVGKQKGVPQSTIDAAGGKVFTFGSYALGVHGPSSDIDTLVVAPKFVTIDEFFQTFPPTFKEMSRVEDIKEFVPVEDAFVPIIKMEYRGVSIDLLFASLPRMASIPKDMDTIDKKNLEGLGESATRSVNGTRVTKELLAAVPQNNSFRHALRAIKLWSSRRGIYGAVFGYPGGVAWAIMVARICQLYPFANGATIVSKFFSLMYKWTWPRPVMLKHIEEGAMGLRVWNPQIYGGDRAHLMPIITPAFPSMCATHTVMPSTLRIMKEEFGRADKILQHVFAGTKKWDALFERHSFFTKDHKYYLSVVAASRTKEANSTFSGLVQSKIRHIVKGIDDGQTGVDIARPYIDYFERYHRCKDEDQIFQVCQGKLDHMIPASELPAEGTAPANGDSHIMYTTTFYIGLTLPLGTATEGTKSLDISYPVSQFKNFITDADKFDKETMSVKVVHTRSSALPDDVFVQGETRPKKPVKEKKKKEAKSAKRQFADTGLDVRDSRLTKPSQRSSASRE

SEQ ID NO: 119 - protein, 546 aa, Drechslerella brochopaga
MPASVDVLKRLQKITEEFVRQVYTSKNQNELTTNSAGGKVFTYGSYRLGVVGPGSDIDTLVVAPKLVTREDFFKFYPPLLKALNKANEPPVIEELAPVPDAFVPIIKFVMSGISIDLIFCRLGVAQVPADMTLEDKNLLRGLDEKELRSLNGTRVTDEILTLVPVPAVFKHALRAIKIWAKCRAIYGNVYGFPGGVAWAMLVARICQLYPSAVSAVIVSKFFRILGQWNWPQPVLLKPIEDGPLNVRVWNPKLYPSDRNHLMPIITPAYPSMCSTHNITPSTKAVILGEMAKAADIVDRIITGNGNWNQLFQRHTFFTKDYKYYLTVKASARDQEQSVKWSGLVESKIRHLVMKLEMLSDVIASARPYVKPFEKVQYCRNQEEAHKIAMGQHVPDSPPAEAANAATGETAGEVSEQEKEGGVPVWITTFHIGIELTPGMVGQVNISWPAQEFYIMCHNWEGYEENMHCVKLELLRNWNLPDECFDFSKGDAKPSRPIVKRKVIRKVVSSSEVSQSGMKRTHAEENPNADEGDVKRTKVNAPVTVQG

SEQ ID NO: 120 - protein, 632 aa, Magnaporthiopsis poae
MAERQLGVTPPVSVSLPSEFELQATDALLAELRAQNSFESPLETQKRHRVLASLQTIADAFVKVVAQEREPHNAVLIKDARAKVFAYGSLRLGVWGPGSDIDTLVVGPRYVTREDYFKHFPDLLVKMAPPGAITDLAVVQEAFVPIIKFEYHGISIDLIFSRIATLKQLPSDPAWDLKDNNLLRGLDEKELRSVNGTRVTDEILSLVPEQNIFRTALRAIKLWAQRRAVYGNIMGFPGGVAWAMLVARVCQLYPKATSSVIVNKFYSILLQWPWPQPVLLKPIGDGPLQVRVWNPRLYKGDSYHLMPVITPAYPSMCATFNVTRSSMTVIYRELQLAAEVTNNIMCSTKPWKDLFTKHTFFTKDFKYYMQVISASRDKEAHKIWSGFVESKVRMLVQSLERHDSIAVARPFIKGFERFHRYRNEEQFAQILDGNLQHMVKAGGDASPSQDGIQAKKEDNSSVKAEEDAKVKQENGETPVKQENGVKVKGEESEEAPLASIPEHSPVDTKGDSKAGDEALEMYTTTHYIGLELNPAAKSLDLSFQVNDFRALCHNWEKYKDELSDRCYVNIKDLRNFALPEDLFEPGEVKPVKSVKKRPAAGANATGDKKRPANEDNSNPAKRRQAASVAAAG

SEQ ID NO: 121 - protein, 631 aa, Cryptococcus depauperatus
MLGVTPPISIEAPKPGDIHSSEALMTDLIAMNQFESDSERKIREKLLSNIAQLVVKFVHDVSLKIGMSEKMASESGGRIYTSGSYRLGVHGPGSDIDTICVCPRHIYREHFFGEFQEMLRAWPEVTEITAVTGAFVPVMKTVISGVEVDLLFARVNLPEAGDSLDIEKDEILRGVDDASQRSLNGPRVTDMILNLVPDVTTFRTALRSIRLWARRRGIYSNVLGFPGGVAWALLTARICQLYPNATPSIIVGKFFPIYYQWNWPQPVILKKIENGPPNMQHAVWNPKLDRRDMAHRMPVITPAYPSMCSTHNITASTMSIISKEMLRAMQITDDILREPGASWAPLFEKVDFFSMYKTYVQVVASASTAEGIKDWSGTVEARIRTLVQDLENTDSILTAHPLVGGVSRVFYCTTEEEQAAASQGELTSEMIDRTEEEVTDKEHRNIFTKSFFIGLEIEKKTKEGGGRVLNLFYPSKKFCSMCQSWDRYNEMEMSVILRPAKRSDLPSFVFPDGIPISKKKAKRQQQNGSADASLSDSVEGQGPSKRTKAAPPSPNQASNPRCNGLPVQNGSDPVSSKIGEEVDIKPPPGIENLPPLSNAAMSSFATAAKGVATTQDFNKEGLVVLNQQSTL

SEQ ID NO: 122 - protein, 571 aa, Golovinomyces cichoracearum
MAEKSFGVTAPLSVTPPTESENQASSALIEELKRQNNYENAIETANRQKVLNSLQLITEEFVRQVSRAQGLPANLIKSAGGMVVTFGSYKLGVIGPGSDIDTLIVAPQNVTKEDFFSRFPDLLRSMATEGNITELTAKPDAFAPCITLKYAGIDIDLLFGRVKLSQVPRNLSLLNQNMLRGLNNEEVRSLNGVRVADEILNLVPEPAIFRTALRTIKLWGTRRAIAGNIYGFPGGVTWAILVARICQLYPKATSSTIVFKFFRIMEKWRWPMPVLLKEIDNLNALGLKVWNPKIYGSDKNHIMPIITPAYPEMCTTHNFSLSTKAILEKELKRGGDITDRVMSGKASWKDLFAKHTFFTNGYKYYLSVVSASRNKESQLFWSGFVESKVRLLVNKLEYHPSIALAHPFNKGFTRVHRCRTEEEVDLVKNGSLKFHATDIATSTTGHGLSVETTSKEKLNETKNVEQDENLIMVYTATHYIGLELAKGAKSLDLSYEVEEFKSICTSSEAYNQEENSLGVAHTKNCDLPDDVFTEGELKPTRPLKQKKKRPATEDSNPVPSKRQQTSLIAVG

SEQ ID NO: 123 - protein, 600 aa, Hortaea werneckii
MAEKQYGVTPAFSLEPPSPKDLKLNDALLAEFKAQNNFAPQSDTEKREAILKKLEGLLQRMVQEVGRKKGLPQSILEVAGGKVFTYGSYRLGVYGPNSDVDTLMVGPKHVTREDFFEHMPPLIRSAWAEDQIGGLVPVPGIGTPIIKLELEGVDIDLIYSSLQLSSIPKDIELKDDNLLRGLDDTDRRCVNGTRVTNRILELVPQTKTFRLALRAIKLWSSQRAIYGNIVGFPGGVAWAILVARVCQLYPKAAAPLLISKFFFIMKRWNWPKPVFLQHKEETSLQLREWDPIQYRGDGFHLMPILTPAYPSMNTAHTVGPSTKMIIMRELERGENIVNDIYANKRPWKDLFQRHTFFTNAYKHYICVVVAAKNKDAHDNWSGLVNSKLKFLVKGIEDSGGSSVELVQPFNKGFSRVHECQTNEQVEKVLDGSLECQVKETKTTEEGNDTAIANGVAQTDTEGLEVPKADSEARAEGSGTTKLWTTTFYLGIGLKKGATDLDISVPVRNLQGDCTAWADYNPDLHSIKIKHIRNFNLPDDVFKEGEVKPQRPKKKTNAAKAAEATSNKRSFSDTGLDPNADPAKRRQSGNVPTPVANGSVG

SEQ ID NO: 124 - protein, 589 aa, Valsa sordida
MAEIQYGMTPPLSTELPKDPEKRANDALFAELKAQNTYEHATETQKREKVLKSLQSIADVFVQKVAQRVHKESPAIQKSARGRVFTYGSYRLGVFGPGSDIDTLVVAPKYVTREDFFELFPGLLKELAPAGSITDLTAVTDAFVPIIKFEYSDISIDLIFSRIATLTEIPALTTKWDLLDNNLLRGLDDAELRSLNGTRVTDDILKLVPEQTSFRLALRAIKLWAQRRAIYANIMGFPGGVAWAMLVARICQLYPKANGAVIVDKFFHIIRRWPWPQPVLLKNPEDGPLQVRVWNPKVYKGDSYHLMPVITPAYPSMCSTFNVTHSNKAVIQKELDHFADVVQQIMMGKLPWKSLFVKHTFFTKDYKYYIAVIAASTTKENSKIWGGFVESKIRLLVQGLERHSSIRLARPFNKGYERKHRTVSGMNEWHDILDGSLDYVVKDEQVADEKAKTEPGNDAVKTEPTSPAKVKQEEGIESLEIKDDAKETDGKNEQDEEDAEVKPETEDPDSPKDVYTSTHYIGIELHDEHYSWSLPDDVFEAGETKPTRPLKKKAAQPPNGAKRKTPTEGALNSAIDAAKRQKTSTVSAG

SEQ ID NO: 125 - protein, 588 aa, Wallemia mellicola
MAEKQWGITPPISLASPTDLEKQVDAGLIQELKDQNTIESEQESLNREKVLALVQSLVKEFVKRVSIRNGMSESLADQAGGKIFTFGSYRLGVNQPGADIDTLCVVPKHVSREDFFDVFEPLLKTREEVSVCAGVPDAYVPIIKTTISGIEIDFLVARLALATIPDDLELADDNLLKNLDERCVRSLNGSRVTDEILRVVPNVQVFRESLRCIKLWAQRRAIYSNVNGFLGGVAWAMLVARICQLYPNENAGAVIAKFFTILYQWKWPHPVLLKEIEDGPLKVRVWNPKVGSSTLLIYPSDRAHRMPIITPAYPSMCSTHNVGPSTQEVMRQEFKRGMDILDGIHKGTTTWSQLFNKHEFFSKYKHYLQIIASGQTAESQKKWSGAVESKVRQLVSKLELVDGIELAHPFVKGFSEVFICLNDEEVQHAAEYNPSEEVKERSKKPEEYENKEAEDGIHRVYISTFYIGLAIQPRNPETNEKRTLNLTYPTNEFMKLTKLWDQFVEGEMKIFVKNIKASALPDVVFEGGIRTSSSKSSKKKSKRPSNVMDESNGENEQNKRLKPDNTSNNEQKSEATALAPSAVPTEST

SEQ ID NO: 126 - protein, 587 aa, Xylaria flabelliformis
MAEKTWGITKPISSAPPTPAESHATALLLEELRRQNTFETASEIKKREKVVNDLQRIADEFVRKVARAKEPHNEVLIRDARGEVFTYGSYCLGVYGPGSDIDTLVTAPRYVTRDDYFKYFPDLLTEMAPPGAITNLTAVEDAFVPIIKFEYWGISIDLIFSRIATLTQFPPHKQLELTSNEHLRGLDDRELRSLNGTRVTKEILNLVPEQSTFRTALRAIKLWAQRRAIYANIIGFPGGVVWAMMVARVCQLYPKATSATIVGKFFLVMKGWPWPIPVQLKHMEDGPLNVRVWNPKIYKSDSFHLMPVITPAYPQMCATFNITKSTKTIIQRELERGVELTDKILGSERPWKDLFVKHTFFTQDHKYYLAVIATSTTKEAHKIWSGFVESKVRILVGELERHSSIALARPFNKGFERQHLTKTDQQAAEVQSGSLAYVTEDEDNSDDATVTKAENGTDGNADSSDQKVTAENTTQPRRTYTTTHYIGLELAEGSKSLDLSYQVNAFKQLCSEWEKYSAEFNSLSVQHVKNINLPEDVFEPGETKPARPLKKPTANGTAPTTQRKRNAPTEGHPPPAKRQQSSAAAAG

SEQ ID NO: 127 - protein, 592 aa, Chaetomium globosum
MSEIIYGVTPPISTTLPTEPEKRLNHALHQELRAQGTFESPQETEKRKEVLRQLEKITTVFVQRAAAQKEPKNTFMIRDAIGRVFTYGSYRLGVYGPGSDMDTLVVAPKYVTVEQYFEIFPEVLVEMAPPGAITDLTPVPEAFVPIIKFEFSGISIDLIFCSIQTLKQLPDEKNWSLADNNLLRGLSENEVRSLNGTRVTDDILHLVPEPATFKLALRAIKLWAQRKAIYANIMGYPGGVAWAMLVARVCQLYPKATSAVVVNKFFHIMFKWPWPLPVLLKDIEYGSPVTRVPVWNPKSSRRAGPKRHRTSMRASSNRGCGCWLTDLRGTILLPWPGPSTRVTTENTDARTIGSWEEVVSVGSLAYVYKPPADSEEKAKAEPKVEPKREVKQEIKAENREPAAAEEKLETRGEDGMRIKQEHSESTQPPPPSNVKPEPHHDTNSEVKLEDIPQKKKDEPDEMEIYTTNHYIGLQLVEGAKSLDLSREVNDWKSMCTSNELFDEGLMFLSIQHLKNTNLPDDVFEPGETKPRPVKKNLKRAASEGPGNMQPQPPVKRHAPGKPSSHSAQQQQQQQQKQRQQRPASTAAAAVAG

SEQ ID NO: 128 - protein, 591 aa, Lachancea thermotolerans
MNQQKTYGVTGPISTAGPTAAENQLNDALIQELKKEKSFESEEDTKKRVEVLRILQNLAQEFVYQVSKKRNMSDGMAKDAGGKIFTYGSYRLGVHGPGSDIDTLVVVPKHVTREDFFTVFDQILRTRSELEEIAPVPDAFVPIIKIKFSGISIDLICARLDIAQVPVNLTLADKNLLRNLDEKDLRALNGTRVTDEILQLVPKPTSFKIALRAIKLWAQRRAVYANIFGFPGGVAWAMLVARICQLYPNACSAVILTRFFHILTKWNWPQPVLLKPIEDGPLQVRVWNPRIYAQDRSHKMPVITPAYPSMCATHNISESTKKVILAELERGAQISSEIFSNKKTWSDLFQKHDFFYKYKFYLTVMASTSGSSEQHLKWSGLVESKLRLLVQKLETLGGINLAHPFTKPFEASYVYDNEAQCKDIIDNYGTHKAQDILAQYTEVTDDNKDTDGVKDKAQVHITTMYIGLDVALDGKKQVDIHVPCSDFFNLCRSFSEYDDTDMFSLMIKYVKLYDLPDNVYVEGEERPVKHSKRKKLGKDSKKSKRPKSNSLKAEEPTSGTTNSEKNNSQGPNKPVKHEPPMQTSPNPLAGS

SEQ ID NO: 129 - protein, 558 aa, Schizosaccharomyces octosporus
MSVKQWGITPPISTAPATEQENALNTALIDELKRQNLFESSAESERRIKVLDDLQRIATEFVKKVSLAKHMNEKMANEAGGKIFTYGSYRLGVYGPGSDIDTLLVVPKHVSRENFFQDLEPMLREREEITELASVPDAYVPIIKFKYFDISIDLIFARLSVPKVPRTLELSDNNLLKGVEERCILSLNGTRVTDQILQLVPNRAVFKHALRAVKFWAQRRAIYANVIGFPGGVAWAMMVARICQLYPNAVSSVIVAKFFRILHQWNWPQPILLKPIEDGPLQVRIWNPKLYPSDKAHRMPIITPAYPSMCATHNITPSTQAIILSEMVRAGEIADQIMVGSVGWSALFQKHDFFHRYKHYLMITAASKTAEGQLKWSGLVESKLRHLVTRLELVEAIALAHPFNKGFDKIHNCKSEEEAHQLASGISLDVASQTADLERLAADANGQNENEDEKEEKKIYPVYTTTFYIGLELEKKKGQPIKKLDISWPAQEFYELCKKWDRYDETQVNVFIKNTRNVNLPDEVFEPGEEKPKSSKKRSYNDAGNNNETMKRQKISTA

SEQ ID NO: 130 - protein, 608 aa, Exophiala spinifera
MAEKQWGMTPPMSTALPEPIDTEKTADLIEELKKENNYEPLEATQKRMATLGLLNRVTQEFVREVSRRRRMPPSQIEQFGGKIFPYGSYRLGVFGPGSDIDTLAVAPRHVTREDFFEYFPTVLKRMTAEGDISSLTPVPDSYVPIIKLVLNDIEIDLIFASIASLQTIPKNLTLNDNNLLTGLDQATIRAVTGPRVTDEILSLVPEQKTFRTALRAIKLWAQRRAVYANIVGYPGGVAWAMLVARVCQLYPHAVGATLVDKFFFVMKNWDWPTPVMLKDIEQPKPSQANEFKVWNPALYKGDKKMLMPIITPAFPSMSATYNISKSGKTVILRELERASQITNNIFAGKARWSDLFKKHSFFTADHKYYLGVTASTLNADSAKQWAGLVESKVRIFVMLLEGIPDITLARPFTKGFKRVHKCADETQIREVQKGSMKYKFEETKTVETTDPELVTSNGDGAAVPAANGAKPETDQGAHTVYTYTFYIGIDTTAKGSLNIAPSFQNFKDICEGWASFNRDSHFLTLASVKCWDLPDDVFDAKAGEVKPSRPVKKVAKQVKAEAKSGVRRSINEVEATETNGDAAKRQKLMTPTPTPTPTPAPKPTAAPA

SEQ ID NO: 131 - protein, 626 aa, Scedosporium apiospermum
MADRQLGVTPPISMALPTPAEIEANNAMVEELRKQGIFESKEETDKRNLVLESLQKMCDEFVTRVAQEKDQGLARDARGKIFTYGGFRLGVFGPGSDIDTLVVAPKYVTRDDYFAIFPDLLLEMAPKGAITGLAVVTDAFVPIIKFEYWGISIDMIFSRIAMLKKLPTNTSQFNLTDTALLRGLDETEIRSLNGTRVAHEILDLVPEQSTFQMALRAIKLWAQRRAIYANVMGYPGGVAWAMLVARVCQLYPRATAATIVSKFFCIMRQWPWPQPVLLKHIERAPLGYRIWNPVVYPSDKHHLMPIITPAYPSMNAAYNINRSSMSIIQTELSRADGITDGIVVGQRPWSDLFEKHTFFTADYKYYLAIVSSGITKDAHKKWSGFVESKVRMLVLALDRHDTIALAQAFVKGYDRVHKCNADSEIQKVQQGDLAYMIKEEDAPKEEQTSPVKSETKPDPGANEKNSAGGSNSNGDVAGSADAVKNEEGANGVKAGDENVYIYTTTHYIGLALRPGQKSINLANEVGEFKKLCKGWEKFDHQLNAITVQHLRNSDLPDDVFAEGEVKPRKRVVRKANGTGPSPPVDAKQATNGTTSQVDSKKRAAAETGAAAPAAKRRQPASVVAAG

SEQ ID NO: 132 - protein, 613 aa, Trichoderma citrinoviride
MAEQNYGVTPPISVTLPTEAENRASDALLEELRRQKTFESPSDTEKRHEICNEFVRKVAREREPKNEILIKNARGKVFTYGSFRLGVFGPGSDIDTLIVAPKYVTREDYFNYFPDLLVSMAPPGAITDLTVVKDAFVPIIKFEYSGISIDLIFSRIIQKQIAPDFHSLKDSSLLRGLDEAELRSLNGTRVTDEILELVPEKSTFKLALRAIKLWAQRRAVYANIMGFPGGVAWAMLVARVCQLYPKATMSVIVNKFFLVIGQWRWPQPVLLKPIEGGPLPVRVWNPKVYKGDSFHLMPVITPAYPSMCATFNITRSSMTIIQRELRRGLEISEQIMVGKRPWSDLFVKHTFFTSGYRYYISVVSASKDKEAHKVWSGYVESKIRMLVQKLEQHPSIALAHAFNKGYDRRHLCRNEQEIGQVQEGSLEFMIKDWDDNKLNGAAIKPEKVEGDSKPNGADIIQPEKVEGESKLSDIPVKPEMDEAESKLSEVAIKTETKDGSTESVFPVEVYTTTHYIGLELEEGAKSLDLSYQVDEFKVLCTSWKKYQEDLEHLVSLGVQHVRNFNLPDDVFEPGEQKPQKKSSAKSLANKKRGAPEDNTPPAKRQQASVAAAG

SEQ ID NO: 133 - protein, 598 aa, Aspergillus thermomutatus
MSAPPVRQWGVTPPISTALPTPDELAANDDLISELKAQNNFESPAETERRKQVLQLIQRVTHEFVKVVSRKKGLSPAAVEAAGGKIFTYGSYRLGVYGPGSDIDTLVVGPKHVLIDDFFSDFPPVLEKMAPPGAIEKMTPVPDAFVPIIKLELSGISIDLIFARLIVSSVPLNLDLKNNDYLRGLDEKEVRSLNGTRVTDEILELVPQQKTFRLALRAIKLWAQRRAIYSNIVGFPGGVAWAMLVARVCQLYPHATGSVIVGKFFRIMNKWAWPQPVLLKPIEDGPLQIKVWNPKIYHGDRFHLMPIITPAYPSMCATHNISMSTKAVILRELQRGGDIVDKIFLKQLTWNDLFARHSFFTHDYKYYLSITASSRTKEAESVWSGLVESKIRHLVGALDRKPTIAVAHPFPKGFERVHIISNEEEAEAVKNGSTKYQDKGTKTETTDETKDAAHQAAAQSGVENAEVEPVGENANGDSRIIYTTTYYIGLELKPLEPGASRSLDISTDAQIFKSTCTSWAGYQPGINDLSITHVRNFDLPEDVFQPGEARPTRPKKKVIKKPEAGAQKRGIDSLDDASLPAAKRQVTSNGISSTPTPA

SEQ ID NO: 134 - protein, 579 aa, Sodiomyces alkalinus
MQDRPLGVTPPISTSLPTDAEHAVNESLLEELKRQNTFESPAETAKREKVLEQLQLICNEFVRRVAREREAGSEALIKEARGRIFTYGSFRLGVYGPGSDIDALVVAPKYVTRDDYFKLFPGLLQEIPPTGAITDLAVVSDAFVPIIKFDFLGISIDLIFSRIASLKQLPKDKDWNLKDSNILRGLDEAELRSLNGTRVTDEIISLVPEPSTFRLALRAIKLWAQRRAVYANIMGFPGGVAWAMLVARVCQLYPKATSSVVVNKFFHIMRRWPWPQPVLLKAVESGPLQVRVWNPKLYKGDQFHLMPIITPAYPSMCATYNITKSAMTVIQRELQRGCEITDSVMMSKQPWSDLFVKHAFFTSDYKHYISVITTSTTKEAHKIWSGYVESKVRVLVQGLEQHPSIALAHAFNKGYDRRHKCSSEQEINQVQEGSLDYLLKAGDSSAGDPAEGNKSPRRPADGEHPEITPEATETIVFTTTHYIGLELVEGAKSLDLSYQVDSFKQLCGQWEKFNSNLNYLSVQHVRNVKLPDDVFEPCETRPQKKSATNGSLQKKKRGASEVDMHPPAKRQQSSVTAAG

SEQ ID NO: 135 - protein, 606 aa, Neohortaea acidophila
MDPKLGVSGTMSMEPPSPKDIKLDDALLQELKSRNEFEAPEETQRRHSVLDRLESVLKRLVAMVGKQQGLPPGILNEAGGKIFTFGSFELGVYGPKSDMDTLMAAPKHVSREDFFQYMPDLLRKEFKPEEIAELTPVPGISVPIIKLELCGVSVDLIFCRLHLQSVPKSQELSNLDLLRGLDDTDLKCVNGTRVTRRILELVPQTKVFRMALRAVKLWAKQRALYGNIVGYPGGVAYAMMVARICQLYPRAAAPLVIWKFFYLMRKWNWPSPVLLQNHEEGSINLREWDPSIYPGDKRHLMPIITPAFPRMNACHTIGPSTKKVLLQEMERAEGIVRSIYESGRPWRDLFQRHSFFTDAYRHYICVITAGRTKEAQQAWSGLVESKVKWLIVGIEHSDAKSVELVQPYNKGFNRVHECKGDADIDKTLDGNLDCQVKEIKTVTTEQAGDVQFQAAAQTDTDGQEVPAPNGEAEVPPQTDGPQTIWTTTFYLGIGLTKGANSLDISSPIRDFTQQCKEWQNYDENLHSIRVKHMRNYDLPADVFAEGETRPTRTKKKSAPKTTDPTAMDAANKKRSFNNSGLDTLDDPAKRRASANGTATPNGVPPR

SEQ ID NO: 136 - protein, 405 aa, Schizosaccharomyces pombe
MNISSAQFIPGVHTVEEIEAEIHKNLHISKSCSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEAPIPPRRQKKTDEQSNKKLLNETDGDNSE

SEQ ID NO: 137 - protein, 333 aa, T. brucei
MPPSPAVVGRSLVNSFKQFVSKDLHTRHVDATYRLVLDCVAAVDPLMRLYTFGSTVVYGVHEKGSDVDFVVLNKTDVEDGKGGDAATQVAKGLQADILAKLARVIRQKHLSWNVEEVRRTRVPVVRVKGGGAVDFDITAYRRNGVRNSALLRAYFEQNPPCRWLSMSIKRWSKQTGLNASVIGGSITSYGFNLMVVYYLLQRNHLQFVPPSTIDVSRVEPLPPHLPLEEPADEGLELGTQVLDFLHFFLHEFDSDKQVISLNRPGITTKEELDWTKSAEDFARMNGEKVHYQWCIEDPYELNLNVGRNVTPLKRDFLRRHLEKARDTALLTIV

SEQ ID NO: 138 - protein, 349 aa, S. pombe
GSHMSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEA

SEQ ID NO: 139 - protein, 480 aa, T. boudieri
MSGPPSGPPSGPRYRGGHRNFGPRHLQSQERQQQQQQQQQQQQQPYQPSPQELAVLAEVVIEQIALATPSAEELERKTKLRERLRKLCTNVSPTAELEAFGSLVSGFATVGSDLDLVLVDKGGSTNRPFFEVPLLLEQELKDNGLEAQLLAKTRVPILKIKQPATEEYPKEVAADIGFANPLAIRNTDMLSMYSKCDPRVIDMVRFIKRWAKRRKINNPYKGTLSSYGYVLMVLHYLINIVEPPVLPNLQLYPIPAETPKDEITTEEGHNVWYYKDVAEIQRRVAEGTMTTNTMDLPLLLLGFFEFYAYRFGWVKDIISIRTKGGLISKVDKGWTVVAVRVGKCEAEYKDRYLFAIEDPFETNHNVSRTCNIYGVRKIRDEFKRANRIMRMREGVGALRYKLFEEAPEDVRPQHHQKQSIAGEEHVNGAPESGREDREGNQLVEELEGHSNGNGIHPSEGYPGPGLAAVVEGVQRLSVDA

SEQ ID NO: 140 - protein, 761 aa, D. stenobrocha
MELTTGDFGGPPGASNATGPGQKPEDPRTMDPVGEQTLESRLRGMLLHGGPPPVDPRTQLRHPSGLAQQAQQAQQQPIGIDPAVPGHLHQLQTAAAPPQQSRLPPSPNIHHQHLQPRLLQRSNTLPPPGGLTQPPPGLERAGTFHGPVPVAQFGYHGSYPTAHSQRGRYGNRRNGFQNAPVPQMNNNLHFPPLGTVPSQPPPPLSERSSLSNMNNPRRHSGPGHYHTDNNHSPPYKRYEAFHGAPGSPQGYSGSPPQNRQGRPYHSSRGSHGGRNNRNFWPLDYDGLTNYAKYIVESVSPTPEEIAMKDNMLRRISEICDKLVPGSRIIPFGSLVSGFATKGADMDVIFAHDTIDPAPSSHESNIPVRLANEFLKRGFEVDLLIKTRVPILKLKTPGEPVSRPGSPVSEGDGDSSEEPWPENVSCDIGFKAHLGITNSHFFRTYSHCDHRFREMVLFVKQWSKNRDLNSPYFGTLSSYGYVLMVAHFLINVVQPPVLPNLQLIPPAAETAESELMQEGFNIWYFKDLERIASGEFLPGGRNEMSLGQLVHEFFQYYTTNFNFVSECVTIRTPGGVMYKQEKGWTSARERVGEMNNTYQDRYLLALEDPFEVSHNVGRTCGGAGVRRIRAEMQRAAHIIRKVSAKEGGTPRSAGWEMPLVVEDLMTTVRETNRGFRNRRVNQKNLMEWIRNDWTVGKCLVQIDAAAEELRKKRELGLPDDAELPPESEEDSEGDEDASEEEEESSSDDGAVRVVGGGSRGGV

SEQ ID NO: 141 - protein, 337 aa, Unknown
  Phytomonas sp.
MTSLSAVNVGKLVCDSFGKLLSKNLDIKPIVQTHEIVSQSMQTVDPSMALYVFGSTAVYGFHESGCDVDFVALNQKDVSDGKAADPSSEIAKGLQVDFLSRLEASLREMHNLAWKMDLVRRTRVPVLRVKGDPCGIDFDVTARRRNGVRNSALLSAYFKQKPETRWLSMAIKQWSKRAGFNMSVDGGCLTSYGCNLMVVYYLLQRQLVKFVDPERCDVAHIPSLPSYLPLEHPAQNGSELGDMVLDFLNYYLHEFNPETEVISLSRADKTTKEMIYWTKQAEDMARISGEKVSYRWCIEDPFEHNLNVGRYVTPFKLTLLRKHMERAKETALLLSIS

SEQ ID NO: 142 - protein, 355 aa, B. saltans
MLSPAAIGKAILDHHASLIGADLSVKRVHDAHSLVKNSLDSVAPDLKLYTFGSSTVFGFHEPKSDVDFVALRQEDIVDGKGGDSTSQLAKGLQTQVLAKLAASVRQKNVQWAVEEVRRARVPVVKVKAPHIDFDITAHRRNGVRNSALLRHYLTQVPENRWLSIAIKSWSKRVGMNGPVGGYLTSYGFNILVVYYLLHRRRHEQPEVEQKEGQDGAASASVDGKQQKELTFIDKDTLDVSLIPPIPEYLALEPPNPETLGEQVLDFFDFYLSRFPMESHVISLSHKEPITKQSLNWTKTAEDMKNNTSMEKVFYRLCIEDPYEVNLNVGRNVSPFKFDLMKKHFVKGRATALGLL

SEQ ID NO: 143 - protein, 425 aa, A. deanei
MPPNPRQLGAAMIEQFRPLLQAPQQSQLTVLSEAPLFQRIQREVHELDPGARVYIFGSTRVYGFHDSGAYQQQSAQVPPSDVDMAVLRREDLLDGSGVDPSSELTRNVQAEFLEKLLNRLQQSEGSASIRSNCSSLVKRTRVPVLRVKHLVSNHSPPHDKEDNISVQVPFDFDITCGRRCGIRNSALFFHYVQQMPMIRFLLLSVKKWSKQTGLNSAIKAPYSNNNNQNHNLTSVMGGSLTSYGFHVLLLYYLLRRGVVQYIPMTVSSAAGSTDRIPNPQLDVNAIPPVPTFLPLLDPALEPHGPQGDPVRYLGELALDWCHFYLHEFDVEKEVVSLSRFDDYFADSPPSGPFPVITKDVLGWTKQREDQGRLRGEKVHYTLCIEDPYEVNLNVGRNVTSLKWMLFRKHLEKAIQTGLLLCPPSK

SEQ ID NO: 144 - protein, 432 aa, P. lactucaedebilis
MPSSSPLGATLPNSSSLAVSAAAAVVPDPLFAFTQFAVDAVRNNTVSHAELVEKERLRQELEAVLQALQPTARLVVYGSLANNLAIANCDMDLMDALHDVSVDLPALYTEALNSQGYSVKLLSKTRTPIVKVSLEHMAVPYCVDISFDNALALHNTKLIATYAACDSRVPILLMFVKLWTAVRRINDPHSGTLSSYGYALLLIFYLQNRCNSPPVLPNLQLISAMGTRSADELECSGYDIWFFKDVEKIQQAQVVKNTSSIGTLLEGFFSYFAYEFDFRDLCISIRTPGGIVTKTAKTWTQMVEHLNDRGDAKVKDRYILSIEDPFEIVHNVGRSVTRAGLYEIRGEFMAATRYVRGNKLDEILNPHRREIVAPTSSTAMHSPQQQHGTQQVSPGEGSSPTPPAVHAEARPSTGTATSTKTSQQAETVVKAE

SEQ ID NO: 145 - protein, 437 aa, S. culicis
MPPPTPMQLGRAILRHYAPSIVVPTAATSGPISSAAASTASLASIHPLFEFIQGCARRVDPQTHLYVFGSTRVYGFSEVSAPTSVGPPAALARPVKNDVDIAALSAADVATTPSPHSADAVVDKGSELAKSLQIDFLEKLKMQLQTQRQHQGDGAPPAASLSWEMEVVKRARVPVLRLQPRPAYTTSAAGGVPTYNVDVTYGRRCGVLNSALLRRYMDQQPDLRYLCLAVKRWSKLTGLNTATSPDGGGLTSYGFHLLLVYYALRRRLVAYVAPENIQWGDIAPVPAALPLRFPGDADGADAWRGRSLGERIAQDDAAAARVGEWALDFVRFYLHEFDYERDVASLSRGAAAAGLVTTEALQWTRQEEYAARGRGEYLFYRFCIEDPYEINLNVGRHMSSVKLMIFKKHLEKALETGLAFIPADEKAKQRVKPKGKE

SEQ ID NO: 146 - protein, 467 aa, B. meristosporus
MSLETDRIVSEFKDLAISQTSETNQPVPDVKVSSVEDSQHHSKAHPPKNENGNNQGHRHGKKPFHRGSTFGEQGKSDRSYGARSFHPNRGTGQYEGKRVLDKSDPRYLKARARFISVLTEQVSELYLKLTPSVEEVSRREQLYFRIHSICEELFPDAQLFQFGSTANGFGLKNADVDFCLCTAENSSLTAVAFVEQLGSVLKERGMENVMLLTRTRIPIIKLKDPVSELNCDIGFNNRLAVYNTRLLRMYAEVDSRVKEIVAIVKHWAKSRQINEPYLGTLSSYAYVLMIIFVLQQRGVLPCLQQICEGEKKEVLVDNYDAYFFDNITDLPKYWKSQNNESLGELLYEFFRFYASDFSYYGSVVSVRTGQVMSKEEKGWTAEATRESGASHVQDRYWVCIEDPFEVTHNLGRPVNKNSLYTIRGEFMRAANILGGTRTSTILERLCQVYVPEEEPPRAPPAPHERSN

SEQ ID NO: 147 - protein, 354 aa, N. californiae
MKTTERTCTFEFYDELTEEINKIVNKIKAKPNDLKKRENLFNYIKLVAKKIFPNSKAYKYGSIENGFSLTNGDIDICILNNKEFNNTPAECVEILGEKLKEDGINDIKLLIRARVPIIKMKDNKSGISCDIGFQNKLAIQNTKLINAYSKIDDRFKQMVFIIKYWSKMKNINEPYMGTLSSYCFILMIIHFLQIKEPPVLPNLQKIYLDNFIEYEYIDDFNVSFFENIDELHKYWNSKNNESLGELLVEFFKYYANDFPYITGVASIRVGNIITKEEKKWTREHQFEINKSNSVKDRYWFCVEDPFEITHNLGRPVDRKSLFRIRGEFLKAVKIINNKNLTALQVLEKLLEKNI

SEQ ID NO: 148 - protein, 344 aa, Unknown
  Perkinsela sp.
MLDPSETCMGRIKNIHAIITSKLMGLLVEKNAQIYCFGSSMVNGVVEKKSDVDVVYLTREDLQKPLSIGDVCNPQSRSEQTSILSAISKILMKDSELFSTVQEKPRARVPYVRGVLKNGLEIDISAHRRNGVRNSLLLRSYFSQIPPTRPSLPNATTSVYRMLSLALKFWGKRTGLVDPVQTFLTSYAFNVLICYYLQQRGGMDFIHPESILIPKGHPTVPDYREIALAGTQGHTCLGWYMRDFLKFYNHEFDYNNTVVSLSRKGITTKEYLGWGLRDEERMHGTDGNAFFYRFCVEDPYENRLNLGRFVTPLRYSMFRMALHQAQLNGFGYLNLKNYGAKIVD

SEQ ID NO: 149 - protein, 352 aa, S. complicate
MMAHPSQGGAHPLSAGPTNPQPYQDAYSGFTEYVLSINRPQYPSANEMYLKEQLRSLIDVEIVQRIQPSARVIPFGSLVNGFATANSDLDLCILDDSPNPRYKIKTELPEVLAAEFERYGFEVKLLTKTRIPIIKLVQGPTAQFPLGLAMDIGFENRLALHNTRLLATYSRIDSRLREMVLFVKHWAKVRGINSSYHGTLSSYGYVLTILHFLINVASPSCLPNLQHIGAQVPVPFEELECEGYNIWFFKDLTNIPPSLNRRSIGELMAEYFSYYAQFDFRNMVISIRTPGGLLTKTSKGWTQALDRVGPEDQKVRSRNFLCIEDPFEITHNVGRTVGKNGLYDVWPFVSFA

SEQ ID NO: 150 - protein, 388 aa, S. ochraceum
MATVFAQSHSLLNTRQPHQHVQQPAQQPPQQQQQQQSTRPKQLSKPRFYAEFSQCLFDFVIQLLPTPEELAIKEDVRKLLERLIRTLEPDSRLLSFGSTANGFSLRNSDMDLCCLIDSDDRLAASDLVTMLGDLLERETKFHVKPLPYARIPIVKLSLDPSPGLPFGIACDIGFENRLALENTRLLMCYAMIDPARVRTMVLFLKVWCKRRKINSPYKGTLSSYGYVLLVIFFLVHVKNPPVLPNLQSMPPLRPMDKEESTLNGHNVWFFDDINLLRQRWQSTNTESVAELLVDFFKFYSREFPYNSGVVSIRAGGIKKDSKGWFSEAERGSARERNRLCIEDPFETDFNVARCVTRDGLYTIRGELMRASRILAARPERPIVALAQL

SEQ ID NO: 151 - protein, 421 aa, G. androsaceus
MAAYSPTMSSNQHYHELNSASPPARRKWLADFSECLFSFVIQLLPTQEELAVKEDVRKLLERLIRTIEPESRLLSFGSTANGYGLRNSDMDLCCLIDSEERLAASDLVTMLGDLLERETKFHVKPLPHARIPIVKLSLDPSPGLPLGIACDIGFENRLALENTRLLMCYAMIDPTRVRTMVLFLKVWSKRRKINSPYKGTLSSYGYVLLVIYFLVHVKNPPVLPNLQQMPPLRPIPKEETYLGGFNTWFFDDIELLRQRWHSENTETVAELLIDFFRYFSRDFPYGVGVASIRAGLLKKDSKGWQNDLSASRYNDARERNRFCIEDPFETDYNVARCVTKDGLYTIRGEFMRASRVLAARPERAILALADLCDERKDEDPCRRASLYSFSSSAPTNTVFGGKSINAFQRNTDYSLSFDQTP

SEQ ID NO: 152 - protein, 512 aa, T. equiperdum
MGVRLYSCDACPHAVFTTHAALLAHAEEHHADLLPDHARLRRIAQKLNPVWNRALNARRNTITSWGKKIFHVAAQRDAGESKMQEAHRARAQLECVVRRWHDKARVFIFGSSVAMGVWDGTADIDFAVVDVDAMERGSWPPLEKNAVRSITELLRRVGFSFVNLEPISHARVPIIKHHASSPILTVARRDAEDVVARSIRFILNGPATREDRLLLEGSVRDAVGPTGVQQVWWNRTSDMMSATLESTTAAVRAAMCSPALASASLRTKVQPAHDECRPELYNIDFALSFRAFGIRNSTLLRKYLLSHPCARPGAIVLKDWSKTSGVNNSVNGYFTSYAINIMWIYYLVQKGYVPYVDPLEIPESLVNYTDFDPRYTPMIDPEITNTEREELYKAAGDMLVGFFYFYSFEFDWGHNVISLNRPGITTKRMLGWHVEDVVPVASTSVSSGGGGSNVKRHPTRYELCIEDPYEENLNLGRHIGVTKSLRVRTELYRGLLSLLKEGETRSCVFAAA

SEQ ID NO: 153 - protein, 531 aa, M. conica
MTAGRDIRSPPRGQRGQGFNKSQNSPRALPLNALDHFPPLGTQPTKAKTPPPNYSQQQQSTTAKPQQRTQYQPLPIDTRNTNPHHARGEVHGRRNFRGDYNQRQLRRDIGPEVCQYLSELSKKVIAEAAPPESEILVKRALLERLEAISKTIVPDAKLIAFGSLVTGFATANSDLDVIFTGGDRVDILNESSDDPSSNFRIPMLLESKLQEEGFGTTLLTKTRVPILKLVQKATEQSPYELQCDIGFSNHLALYNTQMLLTYSKCDPRVKEMMIFIKWWAKRRHINNPYRGTLSSYGYALIVLHYLINVVKPPVLPNLQTFPVPDSAPTNEIIFEGDSDDTFEIWFYKDIEKLPKSDNAMDIGELLKGFFEYYAHNFQWGREVVSIRTKGGLMTKQEKGWVAAVIKPGRTENSEVKNRYLFAVEDPFETEHNVSRTCNGPGVNRIKDEFKRAVWLIRVRDGGKTLFQNLCMEAPPERVWVRREDRDRRGIEEAGGHGSKDHQGAEGEAGDGVKHESVVLSEDGRNALESLV

SEQ ID NO: 154 - protein, 474 aa, P. murina
MDSVKGFEGVESVVFQKEMKKLTKISDFSGKNYGSGNEIEDLKKSLEKMMLESVNIQEKGVFLREKGSEGSYEANLNDKDVLKNNKKDSNFRNIKYYEPKDYGLYVNGKAVWRKNYDEYFVLSTFISHTLKHIMPTNEEISQKERFRFFLSEILNTCRPSAKLVLFGSVASGLAIVNSDMDFCVMDDSLDLHTDEFLKIFSEEIKKYGMETTLLFRTRVSIIKVNSKGSFNFPQGISCDIGFNNKLAIYNTKLLATYSKCDHRVRKIILFVKYWAKRRKINDPYHGTLSSYGYVLLILHYLINIVPIPLLPNLQQMKVASWRSIPQSEIECDGYNVWFYKDIDISCSFSNNTDSLGKLVYGFFYYYAYQFNWKDHVVSIRTQTGLLTKQEKGWTQAMERVFVSDKIFKDRYILAIEDPFEITHNVGRTVNKQSSHIIRGEFFRASKLSSSKLRKKIFNELCEERIIINNFSNVI

SEQ ID NO: 155 - protein, 445 aa, S. japonicus
MSGNGNGHFIPGVHTVEEIERQFVNLALQKRNQAAAAAAAAAERELDPVTCFLLSTYDDVRVSDDELREKDAIMNLLKHVVHSVRPEADIVAFGSIQSGLALKNSDIDACILLPDIGEEMEEFASECFERFTALGFEGKYLRKARIPIIKLLSDTKNRYYYGFQCDIGFNNQLAIYNTSLLHQYSLIDPRCKQLAILVKYWAKQKRINSPYYGTLSSYGYVLMVLFYLIHVVRPAVLPNLQDSPHKQDLYVEGFNVGFVRGTTVARRNTESLPQLLAGFYGFFAHEFNYRESVISIRQPGGLLKKVDKDWTLAKEHTGSADQVIKDRYVLAIEDPFEITHNVGRTVSKAGLFEIRGEFMQATRLLNAATLHSTSAIRKLRASLFKERLEPKSHLKYQKALRQKRMGTEGKAAAEGKTNNPTAASGSDSAPSESASRTATVESREP

SEQ ID NO: 156 - protein, 620 aa, A. nigricans
MNPPQQTTGLEDHLRSLIINNQTPPNHSISPAAKIPHKHHPRTSTDSRGFVRSQTIHRPGPAASQPPSNGPNRRFPGEAQRPDHRPPHLRGPSQNRNYWHGAPPPPRQVVPQGLSPPAATNLHHFPPLGAKVAPPPPSLPSYHSVPTAPCHRPENQRPVYQNDNRDSYHYKQRGLYDGYLAEQWRELDKMAKEVLLTATPVDGEKEAKEKFMQDLEAVCQKVEPTAKLIPFGSIVTGFATRGSDIDCVFTSNTDSALQNDKITEPETPFDRHDELVTQLQESGYNAQLLTRTRVPIIKLVRPATPDVPDSVSCDIGFNNFLAIHNTRLLRTYAACDERLVQMVLFIKWWAKRRHINSPYRGTLSSYGYALLIVHYLINIAQPPVLPNLQLFHPPATQTSTELQYEHNGNVCNIWYLKDTSTLPRSANKASIGELLRGFFEYYAFTFRWQNDVVSIRTAGGILTKMEKNWCAARSRPGGREEGQVWEVKDRVDVQHRYLLAIEDPFETTHNVARTCTFNGVGRIKSELKRAMYLIKSRDPGHHRLRTELFEEAPPERPFVRNGKEVVDKLEKVEIVEERAGVAVEIVEERAGVAVEIVEERAGVALEVVEERAGAAVEVDV

SEQ ID NO: 157 - DNA, 18 bp, Artificial Sequence
  primer
  misc_feature (17)..(17): n is an inosie
ccccccccccccccctnt

SEQ ID NO: 158 - DNA, 12 bp, Artificial Sequence
  primer
  misc_feature (11)..(11): n is an inosine
ttttttttttnt

SEQ ID NO: 159 - DNA, 12 bp, Artificial Sequence
  premier
ccctccctccct

SEQ ID NO: 160 - DNA, 15 bp, Artificial Sequence
  primer
ccctcccctccccct

(19) United States
(12) Patent Application Publication
(10) Pub. No.: US 2024/0279700 A1
(43) Pub. Date: Aug. 22, 2024

(54) ENZYMATIC SYNTHESIS OF POLYNUCLEOTIDES USING 3'-O-AMINO-2'-DEOXYRIBONUCLEOSIDE TRIPHOSPHATE MONOMERS

(71) Applicant: DNA Script, Le Kremlin-Bicêtre (FR)

(72) Inventor: Adrian HORGAN, Le Kremlin-Bicêtre (FR)

(21) Appl. No.: 18/568,729

(22) PCT Filed: Jun. 10, 2022
(86) PCT No.: PCT/EP2022/065839

§ 371 Date: Dec. 8, 2023

(30) Foreign Application Priority Data
Jun. 10, 2021 (EP) 21305796.1

(51) Int. Cl. C12P 19/34; C12N 9/12

(52) U.S. Cl. CPC C12P 19/34; C12N 9/1264

(57) ABSTRACT

The invention is directed to improvements to methods of enzymatic synthesis of polynucleotides employing 3′-O-amino-nucleoside triphosphate monomers in which aldehyde scavenging agents which are O-substituted hydroxy lamines or polyhydroxylamines reduce or prevent spurious capping of growing polynucleotide chains. thereby increasing yields of full length product.

### BACKGROUND

Interest in enzymatic approaches to polynucleotide synthesis has increased not only because of increased demand for synthetic polynucleotides in many areas, such as synthetic biology, CRISPR-Cas9 applications, and high-throughput sequencing, but also because of the limitations of chemical approaches to polynucleotide synthesis, such as upper limits on product length, the use of moisture-sensitive monomers and the use of environmentally unfriendly solvents, Jensen et al, Biochemistry, 57: 1821-1832 (2018).

Currently, most enzymatic approaches for both DNA and RNA synthesis employ template-free polymerases that are used to implement repeated cycles of elongating a 3′-O-protected nucleoside triphosphates to an initiator or an elongated strand and deprotecting until a polynucleotide of the desired sequence is obtained, e.g. Hiatt and Rose, International patent publication WO96/07669. Champion et al, have engineered TdT variants that efficiently incorporate into growing polynucleotide strands reversibly protected 3′-O-amino nucleoside triphosphate monomers developed by Steven Benner (Champion et al, U.S. Pat. No. 10,752,887, and International patent publication WO2020/099451; Benner et al, U.S. Pat. Nos. 7,544,794, 8,034,923, 8,212,020, 10,472,383, and Hutter et al, Nucleosides Nucleotides Nucleic Acids, 29(11): 879-895 (2010)). Unfortunately this protection chemistry is subject to several side reactions that have the effect of either converting the amino protection group into a capping group or removing it prematurely, so that there is either a loss of efficiency by the presence of capped species or the generation of incorrect sequences by successive additions of prematurely deprotected monomers. Benner has addressed the latter problem by modifications to monomer synthesis (U.S. Pat. No. 10,472,383), but the spurious capping problem still remains.

In view of the interest in extending the application of template-free enzymatic synthesis of polynucleotides, the field would be advanced if methods were available to obviate the problem of spurious capping of the 3′-amino protecting group in the context of enzymatic polynucleotide synthesis.

### SUMMARY OF THE INVENTION

The present invention is directed to improved methods and kits for template-free enzymatic synthesis of polynucleotides using 3′-O-amino-protected nucleoside triphosphate monomers. Without intending to be limited by a particular theory of operation, the inventors believe that the exposure of reaction mixtures or reagents containing monomers to adventitious or environmental aldehydes allows such aldehydes to convert 3′-O-amino protection groups to 3′-oximes which have the effect of capping the affected strand, thereby lowering yields. The inventors have discovered that significant increases in product yields may be obtained by incorporating at least one aldehyde scavenger in reaction mixtures and other reagents employed in synthesis.

In some embodiments, the invention is directed to methods of synthesizing a polynucleotide, wherein the method comprises the steps of: (a) providing initiators each with a free 3′-hydroxyl; (b) repeating in a reaction mixture until the polynucleotide is formed, cycles of (i) contacting under elongation conditions the initiators or elongated fragments having free 3′-O-hydroxyls with a 3′-O-amino nucleoside triphosphate and a template-independent polymerase so that the initiators or elongated fragments are elongated by incorporation of a 3′-O-amino nucleoside triphosphate to form 3′-O-amino elongated fragments, and (ii) deprotecting the elongated fragments to form elongated fragments having free 3′-hydroxyls, wherein an effective amount of at least one aldehyde scavenger is present in the reaction mixture.

In one or more embodiments, said effective amount of said at least one aldehyde scavenger is delivered to said reaction mixture by at least one synthesis reagent.

In one or more embodiments, effective amount of said aldehyde scavenger is delivered to the reaction mixture by said synthesis reagent comprising a 3′-O-amino nucleoside triphosphate or a template-independent polymerase, or a mixture of both.

In some embodiments, the polynucleotide can have a predetermined sequence.

### BRIEF DESCRIPTION OF THE DRAWINGS

FIG. 1 diagrammatically illustrates a method of template-free enzymatic synthesis of a polynucleotide.

FIGS. 2A-2C show data on increases in product yields as a function of aldehyde scavenger concentration.

FIGS. 3A-3B show formulas of exemplary O-substituted mono- and polyhydroxylamine aldehyde scavengers which may be used in methods of the invention.

### DETAILED DESCRIPTION OF THE INVENTION

The general principles of the invention are disclosed in more detail herein particularly by way of examples, such as those shown in the drawings and described in detail. It should be understood, however, that the intention is not to limit the invention to the particular embodiments described. The invention is amenable to various modifications and alternative forms, specifics of which are shown for several embodiments. The intention is to cover all modifications, equivalents, and alternatives falling within the principles and scope of the invention.

The practice of the present invention may employ, unless otherwise indicated, conventional techniques and descriptions of organic chemistry, molecular biology (including recombinant techniques), cell biology, and biochemistry, which are within the skill of the art. Such conventional techniques may include, but are not limited to, preparation and use of synthetic peptides, synthetic polynucleotides, monoclonal antibodies, nucleic acid cloning, amplification, sequencing and analysis, and related techniques. Protocols for such conventional techniques can be found in product literature from manufacturers and in standard laboratory manuals, such as Genome Analysis: A Laboratory Manual Series (Vols. I-IV); PCR Primer: A Laboratory Manual; and Molecular Cloning: A Laboratory Manual (all from Cold Spring Harbor Laboratory Press); Lutz and Bornscheuer, Editors, Protein Engineering Handbook (Wiley-VCH, 2009); Hermanson, Bioconjugate Techniques, Second Edition (Academic Press, 2008); and like references.

The invention is directed to the use of aldehyde scavengers in the enzymatic synthesis of polynucleotides using 3′-O-amino-nucleoside triphosphates, especially 3′-O-amino-deoxynucleoside triphosphates. As used herein, the term “aldehyde scavenger” includes ketone scavengers. In some embodiments, aldehyde scavengers are agents that react with compounds having chemical groups of the formula R-C(═O)H or R¹-C(═O)-R², where R, R¹ and R² are typically alkyl or aryl. More particularly, in some embodiments, aldehyde scavengers are agents that react with R-C(═O)H or R¹-C(═O)-R² groups on compounds at a sufficiently high rate that such compounds do not react with (or react only negligibly with) the 3′-amine group of 3′-O-amino-nucleotides. As used herein, the term “scavenger” means a chemical substance added to a mixture in order to remove or de-activate impurities or compounds that lead unwanted reaction products. As noted above, enzymatic synthesis may be carried out using a variety of reagents (referred to herein as “synthesis reagents”) that may contain or consist of reactants, wash solutions, deprotection buffers, enzymes, and the like. The term “synthesis reagent” means any reagent used in a synthesis cycle to couple a monomer, particularly a 3′-O-amino-nucleoside triphosphate, to an initiator or elongated fragment, such as, buffers comprising a template-free polymerase, buffers comprising 3′-O-protected-nucleotide monomers, deprotection (or deblocking) buffers, and the like. In various embodiments, an aldehyde scavenger may be a component of one or more of the synthesis reagents. In some embodiments, an aldehyde scavenger may be added to a reaction mixture as a separate synthesis reagent (without other reactants, wash buffers or enzymes). In some embodiments, an aldehyde scavenger is added to a reaction mixture as a component of a synthesis reagent comprising a template-free polymerase. In some embodiments, more than one aldehyde scavenger is used.

As mentioned above, a method of the invention for synthesizing a polynucleotide may comprise the steps of: (a) providing initiators each with a free 3′-hydroxyl; (b) repeating in a reaction mixture until the polynucleotide is formed, cycles of (i) contacting under elongation conditions the initiators or elongated fragments having free 3′-O-hydroxyls with a 3′-O-amino nucleoside triphosphate and a template-independent polymerase so that the initiators or elongated fragments are elongated by incorporation of a 3′-O-amino nucleoside triphosphate to form 3′-O-amino elongated fragments, and (ii) deprotecting the elongated fragments to form elongated fragments having free 3′-hydroxyls, wherein an effective amount of a aldehyde scavenger is delivered to the reaction mixtures by at least one synthesis reagent.

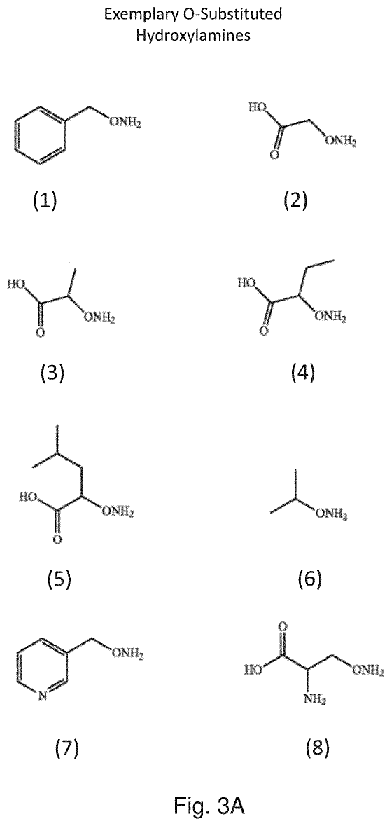

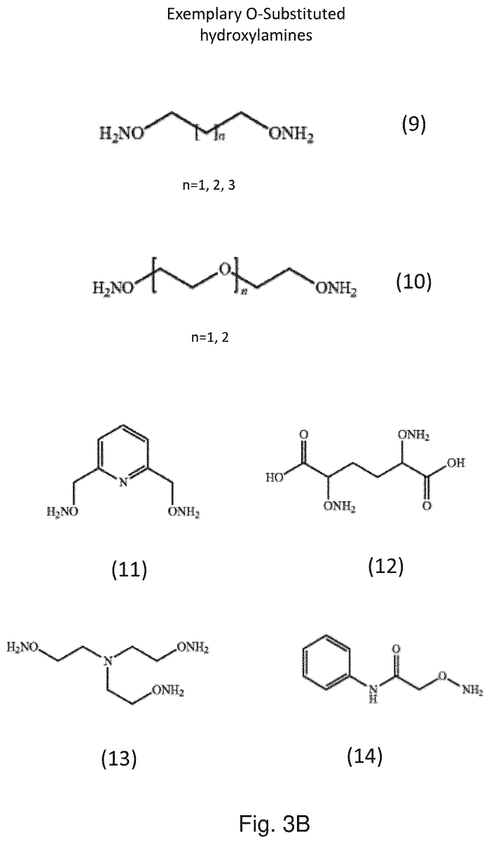

As used herein, the term an “effective amount” means an amount or concentration sufficient to reduce the percentage spuriously capped elongated sequences in a final synthesis product. It is understood that one of ordinary skill could readily determine an effective amount of a particular aldehyde scavenger by conventional techniques, e.g. sequence determination of a sample of polynucleotides from a product. In some embodiments, e.g. employing aldehyde scavengers disclosed by Sudo et al (cited below) or listed in FIGS. 3A-3B, an effective amount is provided by a concentration in the range of from 1 to 500 mM, or in other embodiments in the range of from 1 to 200 mM, or in other embodiments in the range of from 1 to 100 mM.

In some embodiments, aldehyde scavengers employed in the invention comprise O-substituted hydroxylamines or polyhydroxylamines. In some embodiments, O-substituted hydroxylamines used in the invention are defined by the formula:

R₁—ONH₂

such as disclosed by Sudo et al, U.S. patent publication US2020/0061225, or Kitasaka et al, U.S. Pat. No. 7,241,625, which are incorporated herein by reference. In some embodiments, R¹ is a C₁₋₁₈ linear, branched or cyclic alkyl group which may be substituted by at least one substituent selected from the group consisting of a halogen atom; a C₁₋₆ alkyloxy group; a C₁₋₆ haloalkyl group; a C₁₋₆ haloalkyloxy group; a carboxy group; a hydroxy group; a mercapto group; a cyano group; a nitro group; a C₆₋₁₄ aryl group which may be substituted by a halogen atom, a C₁₋₆ alkyl group, a C₁₋₆ alkyloxy group, a C₁₋₆ haloalkyl group, a C₁₋₆ haloalkyloxy group, a carboxy group, a hydroxy group, a mercapto group, a cyano group or a nitro group; a C₄₋₁₄ heteroaryl group which may be substituted by a halogen atom, a C₁₋₆ alkyl group, a C₁₋₆ alkyloxy group, a C₁₋₆ haloalkyl group, a C₁₋₆ haloalkyloxy group, a carboxy group, a hydroxy group, a mercapto group, a cyano group or a nitro group; an alkoxycarbonyl group represented by the following formula:

—(C═O)—O—R₂

and a carbamoyl group represented by the following formula:

—(C═O)—NR₃(R₃)

wherein R² is a C₁₋₁₈ linear, branched or cyclic alkyl group which may be substituted, at a chemically acceptable optional position, by at least one substituent selected from the group consisting of a carboxy group; a hydroxy group; a mercapto group; a halogen atom; a C₁₋₆ alkyloxy group; a C₁₋₆ haloalkyloxy group; a C₆₋₁₄ aryl group; and a C₄₋₁₄ heteroaryl group; and wherein each R³ may be the same or different and each independently a C₁₋₁₈ linear, branched or cyclic alkyl group which may be substituted by at least one substituent selected from the group consisting of a carboxy group; a hydroxy group; a mercapto group; a halogen atom; a C₁₋₆ alkyloxy group; a C₁₋₆ haloalkyloxy group; a C₆₋₁₄ aryl group; and a C₄₋₁₄ heteroaryl group; a C₆₋₁₄ aryl group, a C₄₋₁₄ heteroaryl group, or a hydrogen atom.

In particular, exemplary O-substituted hydroxylamines or polyhydroxylamines which may be used in the invention are shown as compounds (1)-(14) in FIGS. 3A and 3B, wherein compound (1) is also referred to herein as the “BOX” reagent.

In some embodiments aldehyde scavengers comprise carbonyl compounds disclosed by Pacifici, U.S. Pat. No. 5,446,195 or Burdeniuc et al, U.S. patent publication, US20160369035; which are incorporated herein by reference, and are defined by the formula:

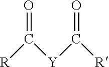

wherein R and R′ are CH3 or H[O(CH2)m]nO— and wherein m and n are selected from the group of combinations of m and n consisting of: m=1 and n=1, 3-19; m=2 and n=2-19; or m=3 and n=1-19, Y is —CH2—or —CH2 —CO—CH2—.

In various embodiments, aldehyde scavengers of the invention may be in solution, immobilized on the materials used for storage or synthesis or coupled to reagents employed in method of the invention, for example, template-free polymerases, such as TdTs.

### Template-Free Enzymatic Synthesis of DNA

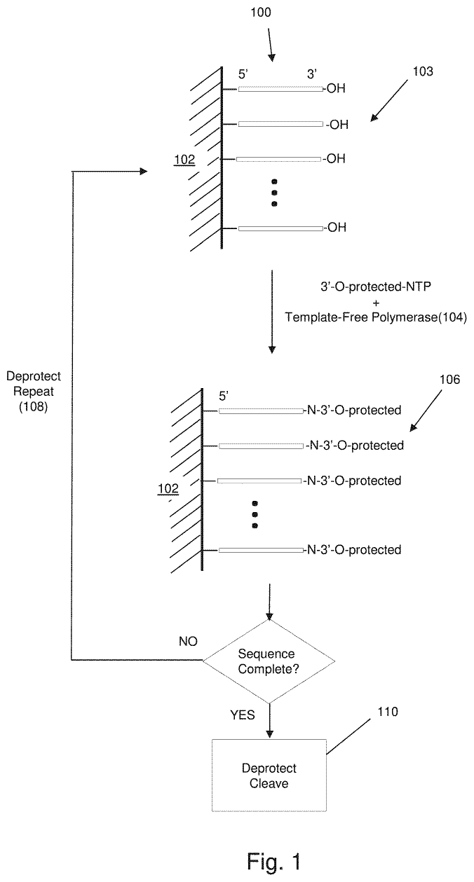

Generally, methods of template-free (or equivalently, “template-independent”) enzymatic DNA synthesis or RNA synthesis comprise repeated cycles of steps (illustrated in FIG. 1) in which a predetermined 3′-O-protected nucleotide is (i) coupled to an initiator or growing chain in each cycle and (ii) deprotected. The general elements of template-free enzymatic synthesis of polynucleotides are described in the following references: Champion et al, WO2019/135007; Hiatt et al, U.S. Pat. No. 5,763,594; and Jensen et al, Biochemistry, 57: 1821-1832 (2018)).

Initiator polynucleotides (100) are provided, for example, attached to solid support (120), which have free 3′-hydroxyl groups (130). To the initiator polynucleotides (100) (or elongated initiator polynucleotides in subsequent cycles) are added a 3′-O-protected-dNTP or 3′-O-protected-rNTP and a template-free polymerase, such as a TdT or variant thereof usually for DNA synthesis (e.g. Ybert et al, WO/2017/216472; Champion et al, WO2019/135007) or a polyA polymerase (PAP) or polyU polymerase (PUP) or variant thereof usually for RNA synthesis (e.g. Heinisch et al, WO2021/018919) under conditions (140) effective for the enzymatic incorporation of the 3′-O-protected-NTP onto the 3′ end of the initiator polynucleotides (100) (or elongated initiator polynucleotides). This reaction produces elongated initiator polynucleotides whose 3′-hydroxyls are protected (106). If the elongated sequence is not complete, then another cycle of addition is implemented (108). If the elongated initiator polynucleotide contains a completed sequence, then the 3′-O-protection group may be removed, or deprotected, and the desired sequence may be cleaved from the original initiator polynucleotide (110). Such cleavage may be carried out using any of a variety of single strand cleavage techniques, for example, by inserting a cleavable nucleotide at a predetermined location within the original initiator polynucleotide. An exemplary cleavable nucleotide may be a uracil nucleotide which is cleaved by uracil DNA glycosylase. If the elongated initiator polynucleotide does not contain a completed sequence, then the 3′-O-protection groups are removed to expose free 3′-hydroxyls (103) and the elongated initiator polynucleotides are subjected to another cycle of nucleotide addition and deprotection.

As used herein, the terms “protected” and “blocked” in reference to specified groups, such as, a 3′-hydroxyls of a nucleotide or a nucleoside, are used interchangeably and are intended to mean a moiety is attached covalently to the specified group that prevents a chemical change to the group during a chemical or enzymatic process. Whenever the specified group is a 3′-hydroxyl of a nucleoside triphosphate, or an extended fragment (or “extension intermediate”) in which a 3′-protected (or blocked)-nucleoside triphosphate has been incorporated, the prevented chemical change is a further, or subsequent, extension of the extended fragment (or “extension intermediate”) by an enzymatic coupling reaction.

As used herein, an “initiator” (or equivalent terms, such as, “initiating fragment,” “initiator nucleic acid,” “initiator oligonucleotide,” or the like) refers to a short oligonucleotide sequence with a free 3′-hydroxyl at its end, which can be further elongated by a template-free polymerase, such as TdT. In one embodiment, the initiating fragment is a DNA initiating fragment. In an alternative embodiment, the initiating fragment is an RNA initiating fragment. In some embodiments, an initiating fragment possesses between 3 and 100 nucleotides, in particular between 3 and 20 nucleotides, which may be all or partially polyC. In some embodiments, the initiating fragment is single-stranded. In alternative embodiments, the initiating fragment may be double-stranded. In some embodiments, an initiator oligonucleotide may be attached to a synthesis support by its 5′ end; and in other embodiments, an initiator oligonucleotide may be attached indirectly to a synthesis support by forming a duplex with a complementary oligonucleotide that is directly attached to the synthesis support, e.g. through a covalent bond. In some embodiments a synthesis support is a solid support which may be a discrete region of a planar solid, or may be a bead.

In some embodiments, an initiator may comprise a non-nucleic acid compound having a free hydroxyl to which a TdT may couple a 3′-O-protected dNTP, e.g. Baiga, U.S. patent publications US2019/0078065 and US2019/0078126.

Synthesis supports to which initiators are attached may comprise polymers, porous or non-porous solids, including beads or microspheres, planar surfaces, such as a glass slide, membrane, or the like. In some embodiments, a solid support, or synthesis support, may comprise magnetic beads, particle-based resins, such as agarose, or the like.

Synthesis supports include, but are not limited to, soluble supports, such as, polymer supports, including polyethylene glycol (PEG) supports, dendrimer supports and the like; non-swellable solid supports, such as, polystyrene particles, Dynabeads, and the like; swellable solid supports, such as resins or gels, including agarose. Synthesis supports may also form part of reaction chambers, such as, the filter membrane of a filter plate. Guidance for selecting soluble supports is found in references Bonora et al, Nucleic Acids Research, 212(5): 1213-1217 (1993); Dickerson et al, Chem. Rev. 102: 3325-3344 (2002); Fishman et al, J. Org. Chem., 68: 9843-9846 (2003); Gavert et al, Chem. Rev. 97: 489-509 (1997); Shchepinov et al, Nucleic Acids Research, 25(22): 4447-4454 (1997): and like references. Guidance for selecting solid supports is found in Brown et al, Synlett 1998(8): 817-827; Maeta et al, U.S. Pat. No. 9,045,573; Beaucage and Iyer, Tetrahedron, 48(12): 2223-2311 (1992); and the like. Guidance for attaching oligonucleotides to solid supports is found in Arndt-Jovin et al, Eur. J. Biochem., 54: 411-418 (1975); Ghosh et al, Nucleic Acids Research, 15(13): 5353-5372 (1987); Integrated DNA Technologies, “Strategies for attaching oligonucleotides to solid supports,” 2014(v6); Gokmen et al, Progress in Polymer Science 37: 365-405 (2012); and like references.

In some embodiments, the solid-phase support will typically be comprised of porous beads or particles in the form of a resin or gel. Numerous materials are suitable as solid-phase supports for the synthesis of polynucleotides. As used herein, the term “particle” includes, without limitation, a “microparticle” or “nanoparticle” or “bead” or “microbead” or “microsphere.” Particles or beads useful in the invention include, for example, beads measuring 1 to 300 microns in diameter, or 20 to 300 microns in diameter, or 30 to 300 microns in diameter, or beads measuring larger than 300 microns in diameter. A particle comprising initiators can be made of glass, plastic, polystyrene, resin, gel, agarose, sepharose, and/or other suitable materials. Of particular interest are porous resin particles or beads, such as, agarose beads. Exemplary agarose particles include Sepharose™ beads. In some embodiments, cyanogen bromide-activated 4% crosslinked agarose beads having diameters in the range of 40-165 μm may be derivatized with initiators for use with methods of the invention. In other embodiments, cyanogen bromide-activated 6% crosslinked agarose beads having diameters in the range of 200-300 μm may be used with methods of the invention. In the latter two embodiments, oligonucleotide initiators having a 5′-aminolinker may be coupled to the Sepharose™ beads for use with the invention. Other desirable linkers for agarose beads include thiol and epoxy linkers.

In some embodiments, a porous resin support derivatized with initiators has average pore diameters of at least 10 nm, or at least 20 nm, or at least 50 nm. In other embodiments, such porous resin support has an average pore diameter in the range of from 10 nm to 500 nm, or in the range of from 50 nm to 500 nm. In some embodiments, initiators are attached to planar supports for massively parallel synthesis of oligonucleotides, e.g. via inkjet delivery of reagents, such as described by Horgan et al, International patent publication WO2020/020608, which is incorporated herein by reference. In some embodiments such planar supports comprise a uniform coating of initiators with protected 3′-hydroxls, wherein, for example, discrete reaction sites may be defined by delivering deprotection solution to discrete locations. In other embodiments, such planar supports comprise an array of discrete reaction sites each containing initiators, which, for example, may be formed on a substrate by photolithographic methods of Brennan, U.S. Pat. No. 5,474,796; Peck et al, U.S. Pat. No. 10,384,189; Indermuhle et al, U.S. Pat. No. 10,669,304; Fixe et al, Materials Research Society Symposium Proceedings. Volume 723, Molecularly Imprinted Materials—Sensors and Other Devices. Symposia (San Francisco, California on Apr. 2-5, 2002); or like references.

After synthesis is completed polynucleotides with the desired nucleotide sequence may be released from initiators and the solid supports by cleavage. A wide variety of cleavable linkages or cleavable nucleotides may be used for this purpose. In some embodiments, cleaving the desired polynucleotide leaves a natural free 5′-hydroxyl on a cleaved strand; however, in alternative embodiments, a cleaving step may leave a moiety, e.g. a 5′-phosphate, that may be removed in a subsequent step, e.g. by phosphatase treatment. Cleaving steps may be carried out chemically, thermally, enzymatically or by photochemical methods. In some embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage may be accomplished by providing initiators with a deoxyinosine as the penultimate 3′ nucleotide, which may be cleaved by endonuclease V at the 3′ end of the initiator leaving a 5′-phosphate on the released polynucleotide, e.g. as taught by Creton, International patent publication WO/2020/165137.

Returning to FIG. 1, in some embodiments, an ordered sequence of nucleotides are coupled to an initiator nucleic acid using a template-free polymerase, such as TdT, in the presence of 3′-O-protected NTPs in each synthesis step. In some embodiments, the method of synthesizing an oligonucleotide comprises the steps of (a) providing an initiator having a free 3′-hydroxyl (100); (b) reacting (104) under extension conditions the initiator or an extension intermediate having a free 3′-hydroxyl with a template-free polymerase in the presence of a 3′-O-protected nucleoside triphosphate to produce a 3′-O-protected extension intermediate (106); (c) deprotecting the extension intermediate to produce an extension intermediate with a free 3′-hydroxyl (108); and (d) repeating steps (b) and (c) (110) until the polynucleotide is synthesized. (Sometimes the terms “extension intermediate” and “elongation fragment” are used interchangeably). In some embodiments, an initiator is provided as an oligonucleotide attached to a solid support, e.g. by its 5′ end. The above method may also include a washing step after each reaction, or extension, step, as well as after each de-protecting step. For example, the step of reacting may include a sub-step of removing unincorporated nucleoside triphosphates, e.g. by washing, after a predetermined incubation period, or reaction time. Such predetermined incubation periods or reaction times typically may be a few seconds, e.g. 30 sec, to several minutes, e.g. 30 min.

When the sequence of polynucleotides on a synthesis support includes reverse complementary subsequences, secondary intra-molecular or cross-molecular structures may be created by the formation of hydrogen bonds between the reverse complementary regions. In some embodiments, base protecting moieties for exocyclic amines are selected so that hydrogens of the protected nitrogen cannot participate in hydrogen bonding, thereby preventing the formation of such secondary structures. That is, base protecting moieties may be employed to prevent the formation of hydrogen bonds, such as are formed in normal base pairing, for example, between nucleosides A and T and between G and C. At the end of a synthesis, the base protecting moieties may be removed and the polynucleotide product may be cleaved from the solid support, for example, by cleaving it from its initiator.

In addition to providing 3′-O-protected NTP monomers with base protection groups, elongation reactions may be performed at higher temperatures using thermal stable template-free polymerases. For example, a thermal stable template-free polymerase having activity above 40° C. may be employed; or, in some embodiments, a thermal stable template-free polymerase having activity in the range of from 40-85° C. may be employed; or, in some embodiments, a thermal stable template-free polymerase having activity in the range of from 40-65° C. may be employed.

In some embodiments, elongation (or coupling) conditions may include adding solvents to an elongation reaction mixture that inhibit hydrogen bonding or base stacking. Such solvents include water miscible solvents with low dielectric constants, such as dimethyl sulfoxide (DMSO), methanol, and the like. Likewise, in some embodiments, elongation conditions may include the provision of chaotropic agents that include, but are not limited to, n-butanol, ethanol, guanidinium chloride, lithium perchlorate, lithium acetate, magnesium chloride, phenol, 2-propanol, sodium dodecyl sulfate, thiourea, urea, and the like. In some embodiments, elongation conditions include the presence of a secondary-structure-suppressing amount of DMSO. In some embodiments, elongation conditions may include the provision of DNA binding proteins that inhibit the formation of secondary structures, wherein such proteins include, but are not limited to, single-stranded binding proteins, helicases, DNA glycolases, and the like.

3′-O-amino-dNTPs without base protection may be purchased from commercial vendors or synthesized using published techniques, e.g. Benner, U.S. Pat. Nos. 7,544,794 and 8,212,020.

When base-protected dNTPs are employed the method of FIG. 1 may further include a step (e) removing base protecting moieties, which in the case of acyl or amidine protection groups may (for example) include treating with concentrated ammonia.

The above method may also include one or more capping steps in addition to washing steps after the coupling (or elongation) step A first capping step may cap, or render inert to further elongations, unreacted 3′-OH groups on partially synthesized polynucleotides. Such capping step is usually implemented after a coupling step, and whenever a capping compound is used, it is selected to be unreactive with protection groups of the monomer just coupled to the growing strands. In some embodiments, such capping steps may be implemented by coupling (for example, by a second enzymatic coupling step) a capping compound that renders the partially synthesized polynucleotide incapable of further couplings, e.g. with TdT. Such capping compounds may be a dideoxynucleoside triphosphate.

Exemplary reaction conditions for an elongation step (also sometimes referred to as an extension step or a coupling step) comprise the following: 2.0-20. μM purified TdT; 125-600 μM 3′-O-blocked dNTP (e.g. 3′-O-NH2-blocked dNTP); about 1 to about 100 mM aldehyde scavenger (e.g. selected from compounds (1)-(14) of FIGS. 3A-3B); about 10 to about 500 mM potassium cacodylate buffer (pH between 6.5 and 7.5) and from about 0.01 to about 10 mM of a divalent cation (e.g. CoCl₂ or MnCl₂), where the elongation reaction may be carried out in a 50 μL reaction volume, at a temperature within the range RT to 45° C., for 3-5 minutes. In embodiments, in which the 3′-O-blocked dNTPs are 3′-O-NH₂-blocked dNTPs, reaction conditions for a deblocking step may comprise the following: 700-1500 mM NaNO₂; 500-1000 mM sodium acetate (adjusted with acetic acid to pH in the range of 4.8-6.5), where the deblocking reaction may be carried out in a 50 μL volume, at a temperature within the range of RT to 45° C. for 30 seconds to several minutes. Washes may be performed with the cacodylate buffer without the components of the coupling reaction (e.g. enzyme, monomer, divalent cations).

### Template-Free Polymerases for Polynucleotide Synthesis

A variety of different template-free polymerases are available for use in methods of the invention. Template-free polymerases include, but are not limited to, polX family polymerases (including DNA polymerases β, λ and μ), poly(A) polymerases (PAPs), poly(U) polymerases (PUPs), DNA polymerase θ, and the like, for example, described in the following references: Ybert et al, International patent publication WO2017/216472; Champion et al, U.S. Pat. No. 10,435,676; Champion et al, International patent publication WO2020/099451; Heinisch et al, International patent publication WO2021/018919. In particular, terminal deoxynucleotidyltransferases (TdTs) and variants thereof are useful in template-free DNA synthesis.

In some embodiments, TdT variants are employed with the invention which display increased incorporation activity with respect to 3′-O-amino nucleoside triphosphates. For example, such TdT variants may be produced using techniques described in Champion et al, U.S. Pat. No. 10,435,676, which is incorporated herein by reference. In some embodiments, a TdT variant is employed having (a) an amino acid sequence at least 80 percent identical to a TdT having an amino acid sequence of any of SEQ ID NOs 7 through 20, inclusive, and 24 through 39, inclusive, and (b) one or more of the substitutions listed in Table 1, wherein the TdT variant (i) is capable of synthesizing a nucleic acid fragment without a template and (ii) is capable of incorporating a 3′-O-modified nucleotide onto a free 3′-hydroxyl of a nucleic acid fragment. In some embodiments, the above TdT variants include a substitution at every position listed in Table 1. In some embodiments, the above percent identity value is at least 85 percent identity with the indicated SEQ ID NOs; in some embodiments, the above percent identity value is at least 90 percent identity with the indicated SEQ ID NOs; in some embodiments, the above percent identity value is at least 95 percent identity with the indicated SEQ ID NOs; in some embodiments, the above percent identity value is at least 97 percent identity; in some embodiments, the above percent identity value is at least 98 percent identity; in some embodiments, the above percent identity value is at least 99 percent identity. As used herein, the percent identity values used to compare a reference sequence to a variant sequence do not include the expressly specified amino acid positions containing substitutions of the variant sequence; that is, the percent identity relationship is between sequences of a reference protein and sequences of a variant protein outside of the expressly specified positions containing substitutions in the variant.

TABLE 1

| SEQ ID | | | | | | |
|---|---|---|---|---|---|---|
| NO | Animal | Substitutions | | | | |
| 1 | Mouse | M192R/Q | C302G/R | R336L/N | R454P/N/A/V | E457N/L/T/S/K |
| 7 | Mouse | M63R/Q | C173G/R | R207L/N | R325P/N/A/V | E328N/L/T/S/K |
| 8 | Bovine | M63R/Q | C173G/R | R207L/N | R324P/N/A/V | E327N/L/T/S/K |
| 9 | Human | M63R/Q | C173G/R | R207L/N | R324P/N/A/V | E327N/L/T/S/K |
| 10 | Chicken | — | C172G/R | R206L/N | R320P/N/A/V | — |
| 11 | Possum | M63R/Q | C173G/R | R207L/N | R331P/N/A/V | E334N/L/T/S/K |
| 12 | Shrew | M63R/Q | C173G/R | R207L/N | — | E328N/L/T/S/K |
| 13 | Python | — | C174G/R | R208L/N | R331P/N/A/V | E334N/L/T/S/K |
| 14 | Canine | M73R/Q | C173G/R | R207L/N | R325P/N/A/V | E328N/L/T/S/K |
| 15 | Mole | M64R/Q | C174G/R | R208L/N | — | E329N/L/T/S/K |
| 16 | Pika | M61R/Q | C171G/R | R205L/N | R323P/N/A/V | E326N/L/T/S/K |
| 17 | Hedgehog | M63R/Q | C173G/R | R207L/N | R328P/N/A/V | E331N/L/T/S/K |
| 18 | Tree shrew | — | C173G/R | R207L/N | R325P/N/A/V | E328N/L/T/S/K |
| 19 | Platypus | M63R/Q | C182G/R | R216L/N | R338P/N/A/V | E341N/L/T/S/K |
| 20 | Jerboa | M66R/Q | C176G/R | R210L/N | R328P/N/A/V | E331N/L/T/S/K |
| 24 | Canary | — | C170G/R | R204L/N | R326P/N/A/V | E329N/L/T/S/K |
| 25 | Neopelma | — | C158G/R | R192L/N | R314P/N/A/V | E317N/L/T/S/K |
| 26 | Alligator | — | — | R205L/N | R327P/N/A/V | E330N/L/T/S/K |
| 27 | Xenopus | — | — | R205L/N | R324P/N/A/V | E327N/L/T/S/K |
| 28 | Tiger snake | — | — | R205L/N | R327P/N/A/V | E330N/L/T/S/K |
| 29 | Brown trout | — | — | R192L/N | R311P/N/A/V | E314N/L/T/S/K |
| 30 | Electric eel | — | — | R205L/N | R321P/N/A/V | E325N/L/T/S/K |
| 31 | Walking fish | — | — | R205L/N | R322P/N/A/V | E325N/L/T/S/K |
| 32 | Guppy | — | — | R205L/N | R322P/N/A/V | E325N/L/T/S/K |
| 33 | Rat | M48R/Q | C158G/R | R192L/N | R310P/N/A/V | E313N/L/T/S/K |
| 34 | Rat | M61R/Q | C171G/R | R205L/N | R323P/N/A/V | E326N/L/T/S/K |
| 35 | Colobus monkey | M61R/Q | C171G/R | R205L/N | R323P/N/A/V | E326N/L/T/S/K |
| 36 | Pig | M61R/Q | C171G/R | R205L/N | R323P/N/A/V | E326N/L/T/S/K |
| 37 | Tiger | M61R/Q | C171G/R | R205L/N | R323P/N/A/V | E326N/L/T/S/K |
| 38 | Water buffalo | M48R/Q | C158G/R | R192L/N | R310P/N/A/V | E313N/L/T/S/K |
| 39 | Marmot | M61R/Q | C171G/R | R205L/N | R323P/N/A/V | E326N/L/T/S/K |

In some embodiments, a TdT variant of the invention is derived from a TdT comprising an amino acid sequence at least 80 percent identical to an amino acid sequence selected from SEQ ID NOs 40 through 75, inclusive , and one or more of the substitutions listed in Table 2, wherein the TdT variant (i) is capable of synthesizing a nucleic acid fragment without a template and (ii) is capable of incorporating a 3′-O-modified nucleotide onto a free 3′-hydroxyl of a nucleic acid fragment. In some embodiments, the above TdT variants include a substitution at every position listed in Table 2. In some embodiments, the above percent identity value is at least 85 percent identity with the indicated SEQ ID NOs; in some embodiments, the above percent identity value is at least 90 percent identity with the indicated SEQ ID NOs; in some embodiments, the above percent identity value is at least 95 percent identity with the indicated SEQ ID NOs; in some embodiments, the above percent identity value is at least 97 percent identity; in some embodiments, the above percent identity value is at least 98 percent identity; in some embodiments, the above percent identity value is at least 99 percent identity. As above, the percent identity values used to compare a reference sequence to a variant sequence do not include the expressly specified amino acid positions containing substitutions of the variant sequence; that is, the percent identity relationship is between sequences of a reference protein and sequences of a variant protein outside of the expressly specified positions containing substitutions in the variant.

TdT variants of SEQ ID NOs 40 through 54, inclusive, 56, 59, 61, 63, 65, 67, 69, 70, 73 and 74 includes substitutions at one or more of the indicated amino acid positions as listed in Table 2 in addition to a stabilizing substitution of the glutamine at position 4 (or a functionally equivalent position). In other embodiments, TdT variants of the invention are derived from natural TdTs such as those listed in Table 2 with a substitution at every one of the indicated amino acid positions in addition to the stabilizing substitution of the glutamine at position 4. In some embodiments, such stabilizing amino acid substituted for glutamine is selected from the group consisting of E, S, D and N. In other embodiments, the stabilizing amino acid is E.

TABLE 2

| SEQ ID | | | | | | |
|---|---|---|---|---|---|---|
| NO | Animal | Substitutions | | | | |
| 1 | Mouse | M192R/Q | C302G/R | R336L/N | R454P/N/A/V | E457N/L/T/S/K |
| 40 | Mouse | M44R/Q | C154G/R | R188L/N | R306P/N/A/V | E309N/L/T/S/K |
| 41 | Bovine | M44R/Q | C154G/R | R188L/N | R305P/N/A/V | E308N/L/T/S/K |
| 42 | Human | M44R/Q | C154G/R | R188L/N | R305P/N/A/V | E308N/L/T/S/K |
| 43 | Chicken | — | C154G/R | R188L/N | R302P/N/A/V | — |
| 44 | Possum | M44R/Q | C154G/R | R188L/N | R312P/N/A/V | E315N/L/T/S/K |
| 45 | Shrew | M44R/Q | C154G/R | R188L/N | — | E309N/L/T/S/K |
| 46 | Canine | M44R/Q | C154G/R | R188L/N | R306P/N/A/V | E309N/L/T/S/K |
| 47 | Mole | M44R/Q | C154G/R | R188L/N | — | E309N/L/T/S/K |
| 48 | Pika | M44R/Q | C154G/R | R188L/N | R306P/N/A/V | E309N/L/T/S/K |
| 49 | Hedgehog | M44R/Q | C154G/R | R188L/N | R309P/N/A/V | E312N/L/T/S/K |
| 50 | Tree shrew | — | C154G/R | R188L/N | R306P/N/A/V | E309N/L/T/S/K |
| 51 | Platypus | M44R/Q | C163G/R | R197L/N | R319P/N/A/V | E322N/L/T/S/K |
| 52 | Canary | — | C153G/R | R187L/N | R309P/N/A/V | — |
| 53 | Neopelma | — | C154G/R | R188L/N | R310P/N/A/V | E311N/L/T/S/K |
| 54 | Alligator | — | — | R188L/N | R310P/N/A/V | E313N/L/T/S/K |
| 56 | Xenopus | — | — | R188L/N | R307P/N/A/V | E310N/L/T/S/K |
| 59 | Brown Trout | — | — | R188L/N | — | E310N/L/T/S/K |
| 61 | Electric eel | — | — | R188L/N | — | — |
| 63 | Walking fish | — | — | R188L/N | R305P/N/A/V | E308N/L/T/S/K |
| 65 | Guppy | — | — | R188L/N | R305P/N/A/V | E308N/L/T/S/K |
| 67 | Rat | — | — | R188L/N | R306P/N/A/V | E309N/L/T/S/K |
| 69 | Piliocolobus | — | — | R188L/N | R306P/N/A/V | E309N/L/T/S/K |
| 70 | Pig | M44R/Q | C154G/R | R188L/N | R306P/N/A/V | E309N/L/T/S/K |
| 73 | Water buffalo | M44R/Q | C154G/R | R188L/N | R305P/N/A/V | E308N/L/T/S/K |
| 74 | Marmot | M44R/Q | C154G/R | R188L/N | R306P/N/A/V | E309N/L/T/S/K |

In some embodiments, further TdT variants for use with methods of the invention include one or more of the substitutions of methionine, cysteine, arginine (first position), arginine (second position) or glutamic acid, as shown in Table 2.

In some embodiments, a TdT variant comprising an amino acid sequence at least ninety percent identical to an amino acid sequence of SEQ ID NOs 55, 57, 58, 60, 62, 64, 66, 68, 71, 72, and 75 through 112, inclusive, may also be used with the present invention.

TdT, PAP and PUP variants for use with the invention each comprise an amino acid sequence having a percent sequence identity with a specified SEQ ID NO, subject to the presence of indicated substitutions. In some embodiments, the number and type of sequence differences between a variant of the invention described in this manner and the specified SEQ ID NO may be due to substitutions, deletion and/or insertions, and the amino acids substituted, deleted and/or inserted may comprise any amino acid. In some embodiments, such deletions, substitutions and/or insertions comprise only naturally occurring amino acids. In some embodiments, substitutions comprise only conservative, or synonymous, amino acid changes, as described in Grantham, Science, 185: 862-864 (1974). That is, a substitution of an amino acid can occur only among members of its set of synonymous amino acids. In some embodiments, sets of synonymous amino acids that may be employed are set forth in Table 3A.

TABLE 3A
Synonymous Sets of Amino Acids I

| Amino Acid | Synonymous Set |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Gly, Ala, Thr, Pro, Ser |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Cys, Ser, Thr |
| His | His, Glu, Lys, Gln, Thr, Arg |
| Gln | Gln, Glu, Lys, Asn, His, Thr, Arg |
| Asn | Asn, Gln, Asp, Ser |
| Lys | Lys, Glu, Gln, His, Arg |
| Asp | Asp, Glu, Asn |
| Glu | Glu, Asp, Lys, Asn, Gln, His, Arg |
| Met | Met, Phe, Ile, Val, Leu |
| Trp | Trp |

In some embodiments, sets of synonymous amino acids that may be employed are set forth in Table 3B.

TABLE 3B
Synonymous Sets of Amino Acids II

| Amino Acid | Synonymous Set |
|---|---|
| Ser | Ser |
| Arg | Arg, Lys, His |
| Leu | Ile, Phe, Met, Leu |
| Pro | Ala, Pro |
| Thr | Thr |
| Ala | Pro, Ala |
| Val | Met, Ile Val |
| Gly | Gly |
| Ile | Met, Phe, Val, Leu, Ile |
| Phe | Met, Tyr, Ile, Leu, Phe |
| Tyr | Trp, Met |
| Cys | Cys, Ser |
| His | His, Gln, Arg |
| Gln | Gln, Glu, His |
| Asn | Asn, Asp |
| Lys | Lys, Arg |
| Asp | Asp, Asn |
| Glu | Glu, Gln |
| Met | Met, Phe, Ile, Val, Leu |
| Trp | Trp |

TdT, PAP and PUP variants for use with the invention are produced by conventional biotechnology technics and may include an affinity tag for purification, which may be attached to the N-terminus, C-terminus or at an interior position of the template-free polymerase. In some embodiments, affinity tags are cleaved before the template-free polymerase is used. In other embodiments, affinity tags are not cleaved before use. In some embodiments, a peptide affinity tag is inserted into a loop 2 region of a TdT variant. An exemplary N-terminal His-tag for use with TdT variants of the invention is MASSHHHHHHSSGSENLYFQTGSSG- (SEQ ID NO: 6)). Guidance for selecting a peptide affinity tag is described in the following references: Terpe, Appl. Microbiol. Biotechnol., 60: 523-533 (2003); Arnau et al, Protein Expression and Purification, 48: 1-13 (2006); Kimple et al, Curr. Protoc. Protein Sci., 73: Unit-9.9 (2015); Kimple et al, U.S. Pat. No. 7,309,575; Lichty et al, Protein Expression and Purification, 41: 98-105 (2005); and the like. Guidance for selecting a peptide affinity tag is described in the following references: Terpe, Appl. Microbiol. Biotechnol., 60: 523-533 (2003); Arnau et al, Protein Expression and Purification, 48: 1-13 (2006); Kimple et al, Curr. Protoc. Protein Sci., 73: Unit-9.9 (2015); Kimple et al, U.S. Pat. No. 7,309,575; Lichty et al, Protein Expression and Purification, 41: 98-105 (2005); and the like.

### Measurement of Nucleotide Incorporation Activity

The efficiency of nucleotide incorporation by variants used with the invention may be measured by an extension, or elongation, assay, e.g. as described in Boule et al (cited below); Bentolila et al (cited below); and Hiatt et al, U.S. Pat. No. 5,808,045, the latter of which is incorporated herein by reference. Briefly, in one form of such an assay, a fluorescently labeled oligonucleotide having a free 3′-hydroxyl is reacted with a template-free polymerase, such as a TdT, under extension conditions for a predetermined duration in the presence of a reversibly blocked nucleoside triphosphate, after which the extension reaction is stopped and the amounts of extension products and unextended oligonucleotide are quantified after separation by gel electrophoresis. By such assays, the incorporation efficiency of a variant template-free polymerase may be readily compared to the efficiencies of other variants or to that of wild type or reference polymerases. In some embodiments, a measure of template-free polymerase efficiency may be a ratio (given as a percentage) of amount of extended product using the variant template-free polymerase over the amount of extended product using wild type template-free polymerase, or reference polymerase, in an equivalent assay.

In some embodiments, the following particular extension assay may be used to measure incorporation efficiencies of TdTs: The primer used is the following:

(SEQ ID NO: 5)
5′-AAAAAAAAAAAAAAGGGG-3′

The primer has also an ATTO fluorescent dye on the 5′ extremity. Representative modified nucleotides used (noted as dNTP in Table 6) include 3′-O-amino-2′,3′-dideoxynucleotides-5′-triphosphates (—ONH2, Firebird Biosciences), such as 3′-O-amino-2′,3′-dideoxyadenosine-5′-triphosphate. For each different variant tested, one tube is used for the reaction. The reagents are added to the tube, starting from water, and then in the order of Table 4. After 30 min at 37° C. the reaction is stopped by addition of formamide (Sigma).

TABLE 4
Extension Activity Assay Reagents

| | | |
|---|---|---|
| Reagent | Concentration | Volume |

| | | | |
|---|---|---|---|
| H₂O | — | 12 | μL |
| Activity buffer | 10x | 2 | μL |

| | | | | |
|---|---|---|---|---|
| dNTP | 250 | μM | 2 | μL |
| Purified enzyme | 20 | μM | 2 | μL |
| Fluorescent primer | 500 | nM | 2 | μL |

The activity buffer comprises, for example, TdT reaction buffer (available from New England Biolabs) supplemented with CoCl₂.

The product of the assay is analyzed by conventional polyacrylamide gel electrophoresis. For example, products of the above assay may be analyzed in a 16 percent polyacrylamide denaturing gel (Bio-Rad). Gels are made just before the analysis by pouring polyacrylamide inside glass plates and let it polymerize. The gel inside the glass plates is mounted on an adapted tank filed with TBE buffer (Sigma) for the electrophoresis step. The samples to be analyzed are loaded on the top of the gel. A voltage of 500 to 2,000V is applied between the top and bottom of the gel for 3 to 6 h at room temperature. After separation, gel fluorescence is scanned using, for example, a Typhoon scanner (GE Life Sciences). The gel image is analyzed using ImageJ software (imagej.nih.gov/ij/), or its equivalent, to calculate the percentage of incorporation of the modified nucleotides.

The elongation efficiency of a template-free polymerase may also be measured in the following hairpin completion assay. In such assay, a test polynucleotide is provided with a free 3′ hydroxyl such that under reaction conditions it is substantially only single stranded, but that upon extension with a polymerase, such as a TdT variant, it forms a stable hairpin structure comprising a single stranded loop and a double stranded stem. This allows the detection of an extension of the 3′ end by the presence of the double stranded polynucleotide. The double stranded structure may be detected in a variety of ways including, but not limited to, (i) fluorescent dyes that preferentially fluoresce upon intercalation into the double stranded structure, (ii) fluorescent resonance energy transfer (FRET) between an acceptor (or donor) on the extended polynucleotide and a donor (or acceptor) on an oligonucleotide that forms a triplex with the newly formed hairpin stem, (iii) FRET acceptors and donors that are both attached to the test polynucleotide and that are brought into FRET proximity upon formation of a hairpin, or the like. In some embodiments, a stem portion of a test polynucleotide after extension by a single nucleotide is in the range of 4 to 6 basepairs in length; in other embodiments, such stem portion is 4 to 5 basepairs in length; and in still other embodiments, such stem portion is 4 basepairs in length. In some embodiments, a test polynucleotide has a length in the range of from 10 to 20 nucleotides; in other embodiments, a test polynucleotide has a length in the range of from 12 to 15 nucleotides. In some embodiments, it is advantageous or convenient to extend the test polynucleotide with a nucleotide that maximizes the difference between the melting temperatures of the stem without extension and the stem with extension; thus, in some embodiments, a test polynucleotide is extended with a dC or dG (and accordingly the test polynucleotide is selected to have an appropriate complementary nucleotide for stem formation).

Exemplary test polynucleotides for hairpin completion assays include p875 (5′-CAGTTAAAAACT) (SEQ ID NO: 2) which is completed by extending with a dGTP; p876 (5′-GAGTTAAAACT) (SEQ ID NO: 3) which is completed by extending with a dCTP; and p877 (5′-CAGCAAGGCT) (SEQ ID NO: 4) which is completed by extending with a dGTP. Exemplary reaction conditions for such test polynucleotides may comprise: 2.5-5 μM of test polynucleotide, 1:4000 dilution of GelRedR (intercalating dye from Biotium, Inc., Fremont, CA), 200 mM Cacodylate KOH PH 6.8, 1 mM CoCl₂, 0-20% of DMSO and 3′-ONH₂ dGTP and TdT at desired concentrations. Completion of the hairpin may be monitored by an increase in fluorescence of GelRed® dye using a conventional fluorimeter, such as a TECAN reader at a reaction temperature of 28-38° C., using an excitation filter set to 360 nm and an emission filter set to 635 nm.

### Kits

The invention includes a variety of kits for implementing a method of enzymatically synthesizing a polynucleotide using 3′-O-amino-nucleoside triphosphate monomers. In one aspect, kits of the invention comprise one or more containers (or bottles, or vials) of synthesis reagents at least one of which contains an effective amount of an aldehyde scavenger. In some embodiments, kits comprise a vial of template-free polymerase and an effective amount of at least one aldehyde scavenger. In some embodiments, the aldehyde scavenger comprises at least one O-substituted hydroxylamine or O-substituted polyhydroxylamine. In some embodiments, the at least one O-substituted hydroxylamine or O-substituted polyhydroxylamine is selected from the group of compounds (1) to (14) of FIGS. 3A-3B. In some embodiments, the at least one O-substituted hydroxylamine or O-substituted polyhydroxylamine is contained in the same vial as a template-free polymerase. In some embodiments, such template-free polymerase is a TdT variant.

In further embodiments, kits may include one or more of the following items, either separately or together with the above-mentioned items: (i) one or more containers comprising 3′-O-amino-dNTPs, (ii) deprotection or de-blocking reagents for carrying out a deprotecting or deblocking step as described herein, (iii) solid supports with initiators attached thereto, (iv) cleavage reagents for releasing completed polynucleotides from solid supports, (iv) wash reagents or buffers for removing unreacted 3′-O-amino-dNTPs at the end of an enzymatic addition or coupling step, and (v) post-synthesis processing reagents, such as purification columns, desalting reagents, eluting reagents, and the like.

### EXAMPLE 1

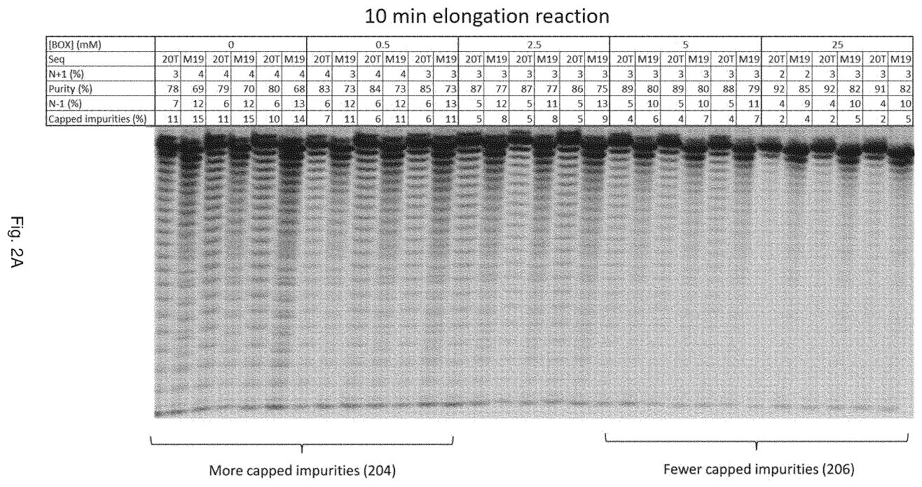

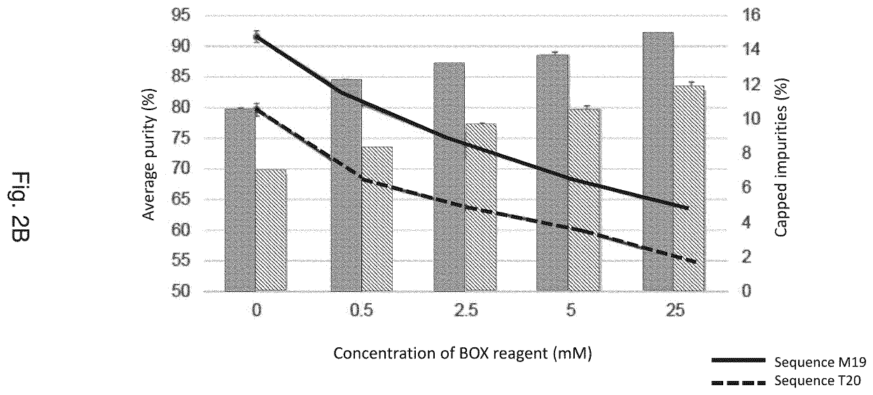

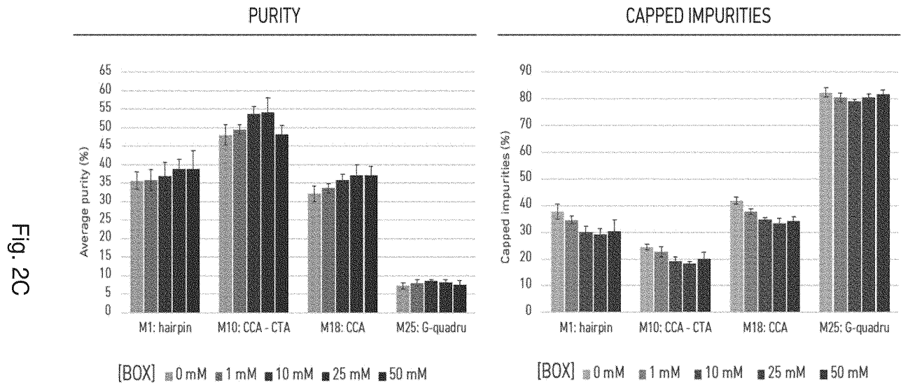

In this example, two test polynucleotides were synthesized as described above, except that the durations of elongation (or coupling) steps were set to 10 minutes. The test polynucleotides were 20T (or a 20-mer polyT) and M19 (ZZZZZZZZZZZZ). Briefly the elongation reaction conditions were carried out on wwPTFE long drip filter plate (PALL), dI fluo resin (internal designation R366—see Creton, International patent publication WO2020/165137), with 500 μM 3′-O-amino-dNTP, 20% DMSO, 20 μM M77 (SEQ ID NO: 106), 500 mM Caco pH 7.4, 2 mM CoCl2, 0.01% tween, 59° C. on heater, V2 deblock. Synthesis reagents were dispensed with 96-head. The BOX (compound (1) of FIG. 3A) was added in enzyme synthesis reagent at final concentration of 0; 0.5; 2.5; 5 or 25 mM. Polynucleotide product was cleaved from supports as taught by Creton (cited above) and analyzed by gel electrophoresis. Results are shown in the electropherogram of FIG. 2A.

The results show clearly by comparison of bands (204—low scavenger concentration) with bands (206—high scavenger concentration) that the fraction of spuriously capped failure sequences drops monotonically with increasing concentration of aldehyde scavenger in the elongation reaction (over the concentration range of 1-25 mM). FIG. 2B shows average purity (bars) and capped impurities (curves), as percentages of product, for sequences 20T (gray bars and solid lines) and M19 (cross hatch bars and dashed lines).

### EXAMPLE 2

In this example, the same synthesis and analysis procedure as Example 1 was followed, except that (i) concentrations of BOX used were 0, 1, 10, 25 and 50 mM, and (ii) different test polynucleotides were used: M1 which contains a hairpin, M10 which contains 3-mer segments CCA and CTA, M18 which contains 3-mer segment CCA, and M25 which contains a G quadruplex. The results are shown in FIG. 2C. The same pattern of increasing purity and decreasing failure sequence percentages with increasing scavenger concentration is shown, at least up to a concentration of about 25 mM.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999).

“Functionally equivalent” in reference to amino acid positions in two or more different TdTs means (i) the amino acids at the respective positions play the same functional role in an activity of the TdTs, and (ii) the amino acids occur at homologous amino acid positions in the amino acid sequences of the respective TdTs. It is possible to identify positionally equivalent or homologous amino acid residues in the amino acid sequences of two or more different TdTs on the basis of sequence alignment and/or molecular modelling. In some embodiments, functionally equivalent amino acid positions belong to inefficiency motifs that are conserved among the amino acid sequences of TdTs of evolutionarily related species, e.g. genus, families, or the like. Examples of such conserved inefficiency motifs are described in Motea et al, Biochim. Biophys. Acta. 1804(5): 1151-1166 (2010); Delarue et al, EMBO J., 21: 427-439 (2002); and like references.

“Kit” refers to any delivery system, such as a package, for delivering materials or reagents for carrying out a method implemented by a system or apparatus of the invention. In some embodiments, consumables materials or reagents are delivered to a user of a system or apparatus of the invention in a package referred to herein as a “kit.” In the context of the invention, such delivery systems include, usually packaging methods and materials that allow for the storage, transport, or delivery of materials, such as, synthesis supports, oligonucleotides, 3′-O-protected-dNTPs, and the like. For example, kits may include one or more enclosures (e.g., boxes) containing solid supports with polyC initiators attached and/or supporting materials. Such contents may be delivered to the intended recipient together or separately. For example, a first container may contain solid supports with polyC initiators attached, while a second or more containers contain a 3′-O-protected-deoxynucleoside triphosphates, a template-free polymerase, for example, a specific TdT variant, and appropriate buffers.

“Mutant” or “variant,” which are used interchangeably, refer to polypeptides derived from a natural or reference TdT polypeptide described herein, and comprising a modification or an alteration, i.e., a substitution, insertion, and/or deletion, at one or more positions. Variants may be obtained by various techniques well known in the art. In particular, examples of techniques for altering the DNA sequence encoding the wild-type protein, include, but are not limited to, site-directed mutagenesis, random mutagenesis, sequence shuffling and synthetic oligonucleotide construction. Mutagenesis activities consist in deleting, inserting or substituting one or several amino-acids in the sequence of a protein or in the case of the invention of a polymerase. The following terminology is used to designate a substitution: L238A denotes that amino acid residue (Leucine, L) at position 238 of a reference, or wild type, sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

“Polynucleotide” or “oligonucleotide” are used interchangeably and each mean a linear polymer of nucleotide monomers or analogs thereof. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

“Sequence identity” refers to the number (or fraction, usually expressed as a percentage) of matches (e.g., identical amino acid residues) between two sequences, such as two polypeptide sequences or two polynucleotide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or ttp://www.ebi.ac.uk/Tools/emboss/. Those skilled in the art can determine appropriate parameters for measuring alignment, including any algorithm needed to achieve maximal alignment over the full length of the sequences being compared. For purposes herein, % amino acid sequence identity values refer to values generated using the pair wise sequence alignment program EMBOSS Needle, that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

“Substitution” means that an amino acid residue is replaced by another amino acid residue. Preferably, the term “substitution” refers to the replacement of an amino acid residue by another selected from the naturally-occurring standard 20 amino acid residues, rare naturally occurring amino acid residues (e.g. hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g. cyclohexyl-alanine). Preferably, the term “substitution” refers to the replacement of an amino acid residue by another selected from the naturally-occurring standard 20 amino acid residues. The sign “+” indicates a combination of substitutions. The amino acids are herein represented by their one-letter or three-letters code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr). In the present document, the following terminology is used to designate a substitution: L238A denotes that amino acid residue (Leucine, L) at position 238 of the parent sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

1. A method of synthesizing a polynucleotide, the method comprising the steps of:
(a) providing initiators each with a free 3′-hydroxyl;
(b) repeating in a reaction mixture until the polynucleotide is formed, cycles of (i) contacting under elongation conditions the initiators or elongated fragments having free 3′-O-hydroxyls with a 3′-O-amino nucleoside triphosphate and a template-independent polymerase so that the initiators or elongated fragments are elongated by incorporation of a 3′-O-amino nucleoside triphosphate to form 3′-O-amino elongated fragments, and (ii) deprotecting the elongated fragments to form elongated fragments having free 3′-hydroxyls,
wherein an effective amount of at least one aldehyde scavenger is present in the reaction mixture.

2. The method of claim 1, wherein said effective amount of the at least one aldehyde scavenger is delivered to said reaction mixture by at least one synthesis reagent.

3. The method of claim 2, wherein said effective amount of said aldehyde scavenger is delivered to the reaction mixture by said synthesis reagent comprising a 3′-O-amino nucleoside triphosphate or a template-independent polymerase, or a mixture of both.

4. The method of claim 1, wherein said polynucleotide is a DNA and wherein said template-independent polymerase is a terminal deoxynucleotidyltransferase (TdT).

5. The method of claim 1, wherein said aldehyde scavenger is an O-substituted mono- or polyhydroxylamine.

6. The method of claim 5, wherein said O-substituted mono- or polyhydroxylamine is selected from the group consisting of compounds defined by the formula:

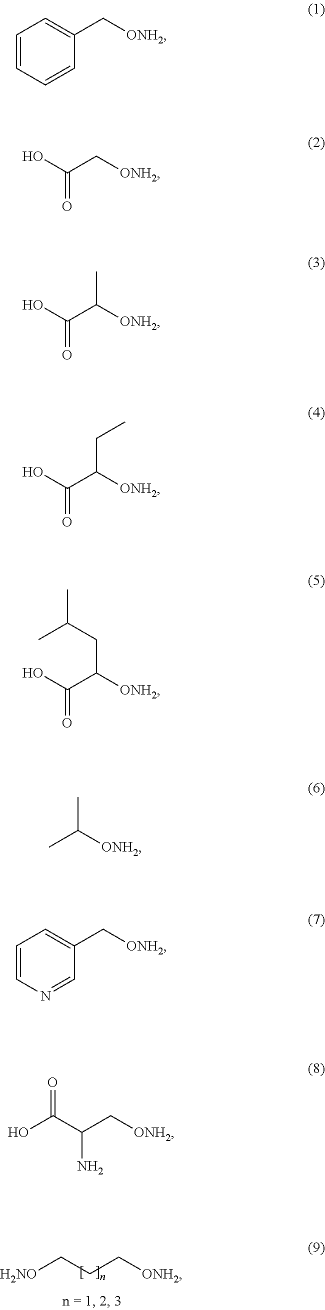

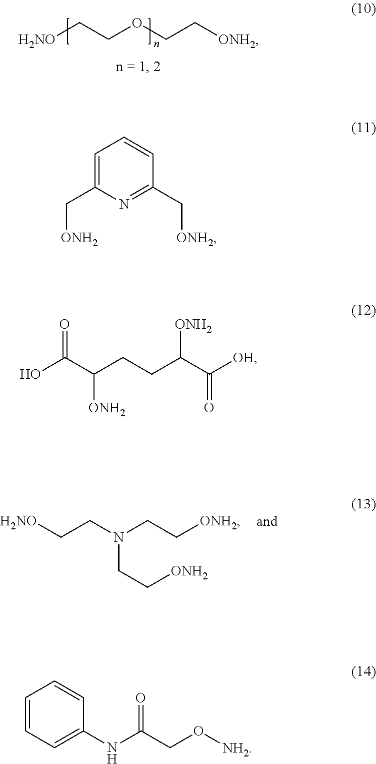

7. The method of claim 1, wherein said effective amount is provided by a concentration of said aldehyde scavenger in the range of from 1 to 100 mM in said reaction mixture.

8. A kit for synthesizing a polynucleotide using a template-free polymerase comprising one or more vials of synthesis reagents each containing an effective amount of at least one aldehyde scavenger.

9. The kit of claim 8 wherein said aldehyde scavenger is an O-substituted mono- or polyhydroxylamine

10. The kit of claim 9, wherein said O-substituted mono- or polyhydroxylamine is selected from the group consisting of compounds defined by the formula:

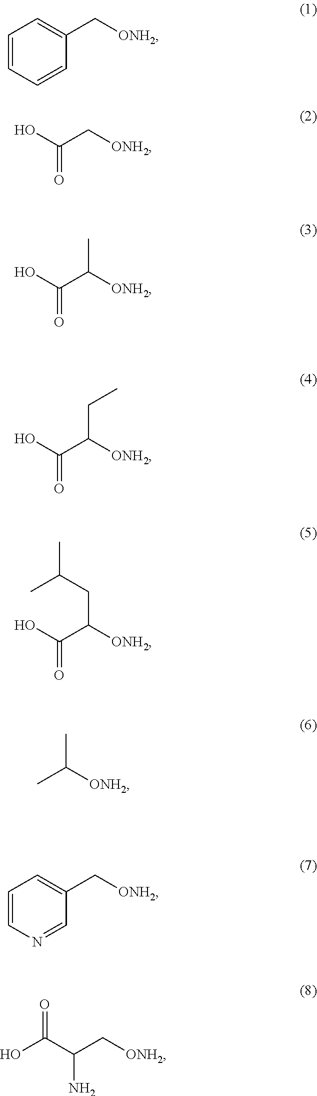

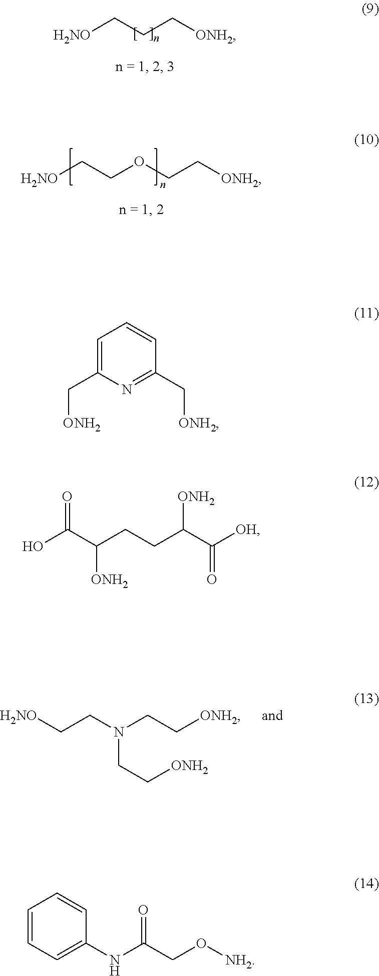

11. The method of claim 1, wherein said aldehyde scavenger is of formula (1)

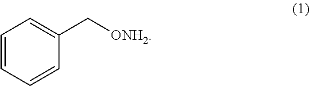

12. The kit of claim 8, wherein said aldehyde scavenger is of formula (1)

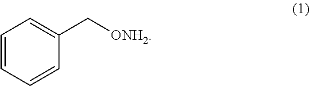

13. The method of claim 1, wherein the total concentration of the at least one aldehyde scavenger in the reaction mixture is about 1 mM to about 500 mM.

14. The method of claim 1, wherein the total concentration of the at least one aldehyde scavenger in the reaction mixture is about 1 mM to about 25 mM.

15. The method of claim 11, wherein the total concentration of the at least one aldehyde scavenger in the reaction mixture is about 1 mM to about 25 mM.
